(19) United States
(12) Patent Application Publication
(10) Pub. No.: US 2019/0184028 A1
(43) Pub. Date: Jun. 20, 2019

(54) TARGETING WITH FIRBRONECTIN TYPE III LIKE DOMAIN MOLECULES

(71) Applicant: Janssen Biotech, Inc., Horsham, PA (US)

(72) Inventors: Vadim Dudkin, Spring House, PA (US); Andrew Elias, Spring House, PA (US); Shalom Goldberg, Spring House, PA (US); Donna Klein, Spring House, PA (US); Elise Kuhar, Spring House, PA (US); Tricia Lin, Spring House, PA (US); Lavanya Peddada, Spring House, PA (US); Karyn O'Neil, Spring House, PA (US); Kristen Wiley, Spring House, PA (US)

(21) Appl. No.: 16/218,990

(22) Filed: Dec. 13, 2018

Related U.S. Application Data
(60) Provisional application No. 62/598,652, filed on Dec. 14, 2017.

(51) Int. Cl. A61K 47/68; A61P 35/00; C07K 14/78; A61P 37/06; B82Y 5/00; A61K 47/69; A61K 47/64; A61K 47/42; A61K 31/7105

(52) U.S. Cl. CPC A61K 47/6807; A61P 35/00; C07K 14/78; A61P 37/06; A61K 31/7105; A61K 47/6929; A61K 47/6925; A61K 47/6415; A61K 47/42; B82Y 5/00

(57) ABSTRACT

A fibronectin type III (FN3) domain-nanoparticle or direct conjugate complex containing a polynucleotide molecule, a toxin, polynucleotide molecule or other pharmaceutically active payload is obtained by panning an FN3 domain library with a protein or nucleotide of interest, recovering the FN3 domain and conjugating the FN3 domain with a toxin or nanoparticle containing an active polynucleotide, such as an ASO or siRNA molecule. A fibronectin type III (FN3) domain-nucleic acid conjugate is obtained by panning an FN3 domain library with a protein or nucleotide of interest, recovering the FN3 domain and conjugating the FN3 domain to a nucleic acid (e.g., ASO or siRNA). The nanoparticle complex, nucleic acid conjugate or FN3 domain toxin conjugate may be used in the treatment of diseases and conditions, for example, oncology or auto-immune indications.

### CROSS REFERENCE TO RELATED APPLICATIONS

This application claims priority to U.S. Provisional Application No. U.S. 62/598,652, filed Dec. 14, 2018, which is hereby incorporated by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “689303.7U1 Sequence Listing” and a creation date of Dec. 13, 2018, and having a size of 58 kb. The sequence listing submitted via EFS-Web is part of the specification and is herein incorporated by reference in its entirety.

### FIELD

The present embodiments relate to targeted delivery of therapeutics using human fibronectin Type III like(FN3) domain molecules and/or FN3 domain molecules that bind to EpCAM. In some embodiments, the embodiments are directed to the use of FN3 domain molecules for delivery of nucleic acid payloads (conjugate) or other pharmaceutically active payloads. Nucleic acids or other payloads are encapsulated in, or associated with, nanoparticles associated with FN3 domain molecules or directly conjugated to FN3 domain molecules to enable cell-specific delivery.

### BACKGROUND OF THE INVENTION

Nucleic acid therapeutics are a new class of medicines with a promise to become the next major therapeutic modality following small molecules, proteins, and vaccines. Nucleic acid therapeutics comprise a variety of oligo- and polynucleotide payloads together with the necessary delivery technology. Multiple nucleic acid payloads have been developed to date to induce gene knockdown (e.g. short interfering RNA, dicer substrate RNA, short hairpin RNA, microRNA, antisense oligonucleotides, U1 adaptors etc), gene editing (e.g., splice-regulating oligonucleotides, CRISPR/CAS) and gene expression or upregulation (e.g., delivery of mRNA, pDNA, mc DNA, etc). It is commonly recognized in the field that successful delivery of nucleic acid payloads into the cytoplasm or nucleus of the target cells is required in order for the modality to reach its therapeutic potential. Targeting such payloads to a cell surface antigen for subsequent internalization is a promising approach to effective intracellular delivery. Short interfering RNA (siRNA) is an example of nucleic acid therapeutic that holds great potential for treating and preventing a myriad of diseases. siRNAs are unique in that they can be designed to match and silence any gene within a cell. Silencing genes can have a significant therapeutic effect in diseased tissues ranging from anti-inflammatory effects to the complete elimination of tumor cells. While pre-clinical data suggest that siRNA will be a powerful new way to treat diseases, in vivo delivery of these siRNA molecules to diseased tissues has been challenging and a major limitation to therapeutic efficacy.

A few key attributes limit in vivo delivery of nucleic acids therapeutics: (1) Poor serum stability, (2) Lack of membrane permeability, and (3) immunogenicity. Owing to these limitations, nucleic acids are often paired with a complementary delivery platform. For example, pairing of nucleic acids, such as siRNA and mRNA with nanoparticles has become a widely adopted strategy for protecting nucleic acid payloads in vivo while improving their delivery to diseased tissues and has seen a significant increase for siRNA clinical trials (Drug Discov Today Technol. 2012 Summer; 9(2):e71-e174.).

While nanoparticle-siRNA complexes have shown some promise in preclinical and early stage clinical trials, their efficacy is limited due to inefficient siRNA delivery to the intracellular space of target cells. A new approach to further enhance delivery of siRNA-nanoparticles is to decorate the nanoparticles with target binding ligands to increase the specificity of the siRNA-NPs and accelerate cellular internalization. Such approaches have proven effective in the delivery of small molecule loaded nanoparticles (Proc Natl Acad Sci USA. 2006 Apr 18; 103(16) 6315-20) and have the potential to provide similar benefits in siRNA delivery.

More recently, clinical candidates employing delivery of mRNA encapsulated in nanoparticles that offer protection from degradation of payloads in systemic circulation have advanced into clinical trials. However, this approach to date has largely been limited to liver delivery, highlighting the need for the next generation of targeting platforms that enable the delivery of mRNA into extrahepatic tissues.

Alternatively, chemically modified single or double stranded oligonucleotide molecules with demonstrated stability in biological fluids may be used for preparing direct conjugates with a targeting domain. Delivery of such conjugates, including siRNA, antisense oligonucleotides, and microRNA mimics and antagonists has been demonstrated using GalNAc, a sugar molecule that specifically binds to the asialoglycoprotein receptor (EPCAM) (J. Am. Chem. Soc. 2014 Dec. 10; 136(49) 16958-16961) or peptides (Nucl. Acids Res. 2014 October; 42(18) 11805-11817). Recently, antibody RNA conjugates have been explored with a series of antibodies that bind and internalize via cell surface receptors (Nucl. Acids. Res. 2014 Dec. 30 ePub)

Ideal targeting ligands for the delivery of nucleic acid-conjugates or nucleic acid-nanoparticle complexes have several key attributes, including, but not limited to, high affinity, high specificity, high stability, efficient and site specific chemical conjugation and small size. Thus, there is a need for an improved process and/or composition to target cells for delivery of nucleic acid-conjugates or nucleic acid-nanoparticle complexes.

### BRIEF DESCRIPTION OF THE FIGURES

The foregoing summary, as well as the following detailed description of the preferred embodiments of the present application, will be better understood when read in conjunction with the appended drawings. It should be understood, however, that the application is not limited to the precise embodiments shown in the drawings.

The patent or application file contains at least one drawing executed in color. Copies of this patent or patent application with color drawing(s) will be provided by the Office upon request and payment of the necessary fee.

FIGS. 1A-1D show flow cytometry histograms demonstrating binding of Tencon25(control)-Superparamagnetic iron oxide nanoparticles (SPION) 83v10-SPION, 83v12-SPION and 83v15-SPION from in H292 cells and the peak to the right corresponding to the 83v10, 83v12 and 83v15 positively shifted compared to the tencon25 control or blank cells.

FIG. 2 shows a quantitative comparison of the binding of all four samples (Tencon25-SPION, 83v10-SPION, 83v12-SPION and 83v15-SPION).

FIG. 3 shows inhibition of EGFR phosphorylation by the FN3 domain molecules coupled to nanoparticles.

FIG. 4 shows the structure of iodoacetamide PEG-MMAF FIG. 5 shows flow cytometry histograms demonstrating binding of an anti-PSMA antibody to the cell lines LNCaP, VCaP, MDA-PCa-2b and PC3.

FIG. 6 shows in vitro cytotoxicity of anti-EGFR Centyrin drug conjugates with 1, 2, 3, or 4 drugs per molecule in NCI-H292 (top) and NCI-H1573 (bottom) tumor cells.

FIG. 7 shows growth kinetics for H292-tumor xenografts following dosing with untargeted or targeted Centyrin drug conjugates or vehicle.

FIG. 8 shows size distribution of AF647-labeled PEG-PLGA-nanoparticles post SEC purification

FIG. 9 shows receptor density dependent and dose-dependent binding and internalization of AF647 labeled 83-Centyrin targeted PEG-PLGA NPs

FIG. 10 shows cellular binding and internalization of AF647 labeled-83-Centyrin (60×) to EGFR-expressing cell line, HCC827

FIG. 11 shows LCMS characterization results for MALAT1 ASO—Centyrin conjugates

FIG. 12 shows MALAT1 gene expression measured by rt-PCR in A431 cells treated with ASO or Centryin-ASO by free uptake.

FIG. 13 shows LC-MS of MALAT1-CD8 368 Centyrin conjugate FIG. 14 shows MALAT1 gene expression measured by rt-PCR in primary T cells treated with ASO or Centryin-ASO conjugates by free uptake.

### SUMMARY

The present embodiments provides compositions comprising FN3 domain molecules to cell associated target ligands, that can be used, for example, for delivery of nucleic acid therapeutic payloads or other payloads and methods of producing such FN3 domain molecules. In some embodiments, the active moiety is a nanoparticle containing a nucleic acid molecule and the FN3 domain molecule is attached to the nanoparticle, either directly or indirectly, such as through a covalent bond. In another embodiment, the active moiety is a chemically modified nucleic acid molecule engineered for covalent attachment to the FN3 domain molecule.

The FN3 domain molecule may be based on a consensus sequence of FN3 domains from human tenascin (from the tencon FN3 domain as described in U.S. Pat. No. 8,278,419, incorporated herein by reference in its entirety, from the stabilized tencon FN3 domain as described in U.S. Pat. No. 8,569,227, incorporated herein by reference in its entirety, or from the tencon molecule with alternative binding surfaces as described in U.S. Pat. No. 9,200,273, incorporated herein by reference in its entirety), or from other fibronectin domains (the consensus FN3 domain as described in U.S. Pat. No. 8,293,482, incorporated herein by reference in its entirety).

In some embodiments, the nanoparticle comprises a cyclodextrin nanoparticle comprising a polymer containing a cyclodextrin or modified cyclodextrin. In other embodiments, the nanoparticle is composed of a polymeric matrix composed of two or more polymers. In yet another embodiment, the copolymer is a copolymer of PLGA or PLA and PEG. In still another embodiment, the polymeric matrix comprises PLGA or PLA and a copolymer of PLGA or PLA and PEG. In some embodiments, the nanoparticle is a lipid nanoparticle or polymeric nanoparticle. In some embodiments, the nanoparticle comprises a liposome, where the liposome bilayer membrane contains a vesicle-forming lipid derivatized with hydrophilic polymer. In another embodiment, the nanoparticle comprises a superparamagnetic iron oxide core coated with a hydrophilic polymer. In another embodiment, the nanoparticle comprises a dendrimer. In further embodiment, the nanoparticle is a solid lipid nanoparticle comprised of at least one lipid and emulsifier.

In another embodiment, the FN3 domain molecule is a cysteine engineered fibronectin type III (FN3) domain (as described in U.S. application Ser. No. 14/512,801, incorporated herein by reference in its entirety).

Another aspect of the invention is a method of targeting a cellular ligand by linking an FN3 domain molecule having binding specificity for a cellular target with a nanoparticle containing an siRNA molecule with therapeutic activity and administering the composition to a subject or patient.

In some embodiments, the nanoparticle is CDP or modified CDP or solid lipid nanoparticle, the FN3 domain molecule targets prostate specific membrane antigen (PSMA) or epidermal growth factor receptor (EGFR) and the siRNA is active against the androgen receptor (AR), EGFR, KRas, or PLK-1. In one specific embodiment, the nanoparticle is CDP or modified CDP or solid lipid nanoparticle, the FN3 domain molecule targets PSMA and the siRNA is active against AR. In another specific embodiment, the nanoparticle is CDP or modified CDP or solid lipid nanoparticle, the FN3 domain molecule targets EGFR or PSMA and the siRNA is active against EGFR, KRas or AR. In yet another specific embodiment, the conjugate is a chemically modified siRNA, the FN3 domain targets EGFR or PSMA and the siRNA is active against PLK-1. In some embodiments, the FN3 domain is a domain that binds to PSMA, EGFR, EpCam, CD22, BCMA, CD33, CD71 and/or CD8. In some embodiments, the FN3 domain contains multiple domains that are specific for different molecules.

FN3 domain molecules are well-suited for conjugation since they contain no cysteine residues. Thus, a unique cysteine can be added to FN3 domain molecules by site-directed mutagenesis and used for site-specific conjugation using simple, well-established chemistry. For nanoparticle targeting or nucleic acid conjugates, site specific coupling is a key advantage as it guarantees the orientation of the targeting ligand which is critical for proper target engagement. For targeted nanoparticles, a blending of the three technologies described here (siRNA, nanoparticle, FN3 domain molecules) is expected to create a highly specific, potent therapeutic agent for gene silencing or gene delivery. For targeted nucleic acid conjugates, a combination of optimized RNA chemistry and FN3 domain molecules is expected to create a highly specific, potent therapeutic agent for gene silencing.

### DETAILED DESCRIPTION

The term “fibronectin type III (FN3) like domain” (FN3 domain) as used herein refers to a domain occurring frequently in proteins including fibronectins, tenascin, intracellular cytoskeletal proteins, cytokine receptors and prokaryotic enzymes (Bork and Doolittle, Proc Nat Acad Sci USA 89:8990-8994, 1992; Meinke et al., J Bacteriol 175:1910-1918, 1993; Watanabe et al., J Biol Chem 265:15659-15665, 1990). Exemplary FN3 domains are the 15 different FN3 domains present in human tenascin C, the 15 different FN3 domains present in human fibronectin (FN), and non-natural synthetic FN3 domains as described for example in U.S. Pat. Publ. No. 2010/0216708. Individual FN3 domains are referred to by domain number and protein name, e.g., the 3^{rd} FN3 domain of tenascin (TN3), or the 10^{th} FN3 domain of fibronectin (FN10).

The term “substituting” or “substituted” or “mutating” or “mutated” as used herein refers to altering, deleting of inserting one or more amino acids or nucleotides in a polypeptide or polynucleotide sequence to generate a variant of that sequence.

The term “randomizing” or “randomized” or “diversified” or “diversifying” as used herein refers to making at least one substitution, insertion or deletion in a polynucleotide or polypeptide sequence.

“Variant” as used herein refers to a polypeptide or a polynucleotide that differs from a reference polypeptide or a reference polynucleotide by one or more modifications for example, substitutions, insertions or deletions.

The term “specifically binds” or “specific binding” as used herein refers to the ability of the FN3 domain of the invention to bind to a predetermined antigen with a dissociation constant (K_{D}) of 1×10⁻⁶ M or less, for example 1×10⁻⁷ M or less, 1×10⁻⁸ M or less, 1×10⁻⁹M or less, 1×10⁻¹⁰ M or less, 1×10⁻¹¹ M or less, 1×10⁻¹² M or less, or 1×10⁻¹³ M or less. Typically the FN3 domain of the invention binds to a predetermined antigen with a K_{D} that is at least ten fold less than its K_{D} for a nonspecific antigen (for example BSA or casein) as measured by surface plasmon resonance using for example a Proteon Instrument (BioRad).

The term “library” refers to a collection of variants. The library may be composed of polypeptide or polynucleotide variants.

The term “stability” as used herein refers to the ability of a molecule to maintain a folded state under physiological conditions such that it retains at least one of its normal functional activities, for example, binding to a predetermined antigen.

“Tencon” as used herein refers to the synthetic fibronectin type III (FN3) domain having the sequence described in U.S. Pat. No. 8,278,419.

The term “vector” means a polynucleotide capable of being duplicated within a biological system or that can be moved between such systems. Vector polynucleotides typically contain elements, such as origins of replication, polyadenylation signal or selection markers that function to facilitate the duplication or maintenance of these polynucleotides in a biological system. Examples of such biological systems may include a cell, virus, animal, plant, and reconstituted biological systems utilizing biological components capable of duplicating a vector. The polynucleotide comprising a vector may be DNA or RNA molecules or a hybrid of these.

The term “expression vector” means a vector that can be utilized in a biological system or in a reconstituted biological system to direct the translation of a polypeptide encoded by a polynucleotide sequence present in the expression vector.

The term “polynucleotide” means a molecule comprising a chain of nucleotides covalently linked by a sugar-phosphate backbone or other equivalent covalent chemistry. Double and single-stranded DNAs and RNAs are typical examples of polynucleotides.

The term “polypeptide” or “protein” means a molecule that comprises at least two amino acid residues linked by a peptide bond to form a polypeptide. Small polypeptides of less than about 50 amino acids may be referred to as “peptides”.

“Valent” as used herein refers to the presence of a specified number of binding sites specific for an antigen in a molecule. As such, the terms “monovalent”, “bivalent”, “tetravalent”, and “hexavalent” refer to the presence of one, two, four and six binding sites, respectively, specific for an antigen in a molecule.

“Mixture” as used herein refers to a sample or preparation of two or more FN3 domains not covalently linked together. A mixture may consist of two or more identical FN3 domains or distinct FN3 domains.

For purposes of the invention, the nanoparticle may comprise a polymeric matrix. In one embodiment, the polymeric matrix comprises two or more polymers. In another embodiment, the polymeric matrix comprises polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes or polyamines or combinations thereof. In still another embodiment, the polymeric matrix comprises one or more polyesters, polyanhydrides, polyethers, polyurethanes, polymethacrylates, polyacrylates or polycyanoacrylates. In another embodiment, at least one polymer is a polyalkylene glycol. In yet another embodiment, at least one polymer is a polyester. In another embodiment, polyester is selected from the group consisting of PLGA, PLA, PGA and polycaprolactones. In another embodiment, the polymeric matrix may consist of CDP or modified CDP PEG polymers. In another embodiment the nanoparticle may comprise lipid molecules. In another embodiment, the nanoparticle may comprise a solid lipid nanoparticle.

### Compositions of Matter

The present invention provides monospecific and multi-specific (e.g., bispecific) FN3 domains with binding specificity to cellular targets and bonded to or CDP or modified CDP or solid lipid nanoparticles containing active siRNA molecules or directly conjugated to chemically modified siRNA molecules.

Isolation of FN3 Domains from a Library Based on Tencon Sequence

Tencon is a non-naturally occurring fibronectin type III (FN3) domain designed from a consensus sequence of fifteen FN3 domains from human tenascin-C (Jacobs et al., Protein Engineering, Design, and Selection, 25:107-117, 2012; U.S. Pat. Publ. No. 2010/0216708). The crystal structure of Tencon shows six surface-exposed loops that connect seven beta-strands as is characteristic to the FN3 domains, the beta-strands referred to as A, B, C, D, E, F, and G, and the loops referred to as AB, BC, CD, DE, EF, and FG loops (Bork and Doolittle, Proc Natl Acad Sci USA 89:8990-8992, 1992; U.S. Pat. No. 6,673,901). These loops, or selected residues within each loop, can be randomized in order to construct libraries of fibronectin type III (FN3) domains that can be used to select novel molecules that bind cellular proteins and nucleotides useful for targeting for active agents, such as CDP or modified CDP PEG or solid lipid nanoparticles containing siRNA.

Library designs based on Tencon sequence may thus have randomized FG loop, or randomized BC and FG loops, such as libraries TCL1 or TCL2 as described below. The Tencon BC loop is 7 amino acids long, thus 1, 2, 3, 4, 5, 6 or 7 amino acids may be randomized in the library diversified at the BC loop and designed based on Tencon sequence. The Tencon FG loop is 7 amino acids long, thus 1, 2, 3, 4, 5, 6 or 7 amino acids may be randomized in the library diversified at the FG loop and designed based on Tencon sequence. Further diversity at loops in the Tencon libraries may be achieved by insertion and/or deletions of residues at loops. For example, the FG and/or BC loops may be extended by 1-22 amino acids, or decreased by 1-3 amino acids. The FG loop in Tencon is 7 amino acids long, whereas the corresponding loop in antibody heavy chains ranges from 4-28 residues. To provide maximum diversity, the FG loop may be diversified in sequence as well as in length to correspond to the antibody CDR3 length range of 4-28 residues. For example, the FG loop can further be diversified in length by extending the loop by additional 1, 2, 3, 4 or 5 amino acids.

Library designs based on Tencon sequence may also have randomized alternative surfaces that form on a side of the FN3 domain and comprise two or more beta strands, and at least one loop. One such alternative surface is formed by amino acids in the C and the F beta-strands and the CD and the FG loops (a C-CD-F-FG surface).

Library designed based on Tencon sequence also includes libraries designed based on Tencon variants, such as Tencon variants having substitutions at residues positions 11, 14, 17, 37, 46, 73, or 86, and which variants display improve thermal stability. Exemplary Tencon variants are described in US Pat. Publ. No. 2011/0274623, and include Tencon27 having substitutions E11R, L17A, N46V, E86I when compared to the base Tencon sequence.

TABLE 1

| FN3 domain | Tencon |
|---|---|
| A strand | 1-12 |
| AB loop | 13-16 |
| B strand | 17-21 |
| BC loop | 22-28 |
| C strand | 29-37 |
| CD loop | 38-43 |
| D strand | 44-50 |
| DE loop | 51-54 |
| E strand | 55-59 |
| EF loop | 60-64 |
| F strand | 65-74 |
| FG loop | 75-81 |
| G strand | 82-89 |

Tencon and other FN3 sequence based libraries can be randomized at chosen residue positions using a random or defined set of amino acids. For example, variants in the library having random substitutions can be generated using NNK codons, which encode all 20 naturally occurring amino acids. In other diversification schemes, DVK codons can be used to encode amino acids Ala, Trp, Tyr, Lys, Thr, Asn, Lys, Ser, Arg, Asp, Glu, Gly, and Cys.

Alternatively, NNS codons can be used to give rise to all 20 amino acid residues and simultaneously reducing the frequency of stop codons. Libraries of FN3 domains with biased amino acid distribution at positions to be diversified can be synthesized for example using Slonomics® technology (http:_//www_sloning_com). This technology uses a library of pre-made double stranded triplets that act as universal building blocks sufficient for thousands of gene synthesis processes. The triplet library represents all possible sequence combinations necessary to build any desired DNA molecule. The codon designations are according to the well known IUB code.

The FN3 domains specifically binding cellular proteins or nucleotides for targeting can be isolated by producing the FN3 library such as the Tencon library using cis display to ligate DNA fragments encoding the scaffold proteins to a DNA fragment encoding RepA to generate a pool of protein-DNA complexes formed after in vitro translation wherein each protein is stably associated with the DNA that encodes it (U.S. Pat. No. 7,842,476; Odegrip et al., Proc Natl Acad Sci USA 101, 2806-2810, 2004), and assaying the library for specific binding to the protein or nucleotide of interest by any method known in the art and described in the Example. Exemplary well known methods which can be used are ELISA, sandwich immunoassays, and competitive and non-competitive assays (see, e.g., Ausubel et al., eds, 1994, Current Protocols in Molecular Biology, Vol. 1, John Wiley & Sons, Inc., New York). The identified FN3 domains specifically binding to the protein or nucleotide of interest are further characterized for their activity.

The FN3 domains specifically binding to the protein or nucleotide of interest can be generated using any FN3 domain as a template to generate a library and screening the library for molecules specifically binding to the protein or nucleotide of interest using methods provided within. Exemplary FN3 domains that can be used are the 3rd FN3 domain of tenascin C, Fibcon, and the 10^{th} FN3 domain of fibronectin. Standard cloning and expression techniques are used to clone the libraries into a vector or synthesize double stranded cDNA cassettes of the library, to express, or to translate the libraries in vitro. For example, ribosome display (Hanes and Pluckthun, Proc Natl Acad Sci USA, 94, 4937-4942, 1997), mRNA display (Roberts and Szostak, Proc Natl Acad Sci USA, 94, 12297-12302, 1997), or other cell-free systems (U.S. Pat. No. 5,643,768) can be used. The libraries of the FN3 domain variants may be expressed as fusion proteins displayed on the surface for example of any suitable bacteriophage. Methods for displaying fusion polypeptides on the surface of a bacteriophage are well known (U.S. Pat. Publ. No. 2011/0118144; Int. Pat. Publ. No. WO2009/085462; U.S. Pat. Nos. 6,969,108; 6,172,197; 5,223,409; 6,582,915; 6,472,147).

The FN3 domains specifically binding to the protein or nucleotide of interest can be modified to improve their properties such as improve thermal stability and reversibility of thermal folding and unfolding. Several methods have been applied to increase the apparent thermal stability of proteins and enzymes, including rational design based on comparison to highly similar thermostable sequences, design of stabilizing disulfide bridges, mutations to increase alpha-helix propensity, engineering of salt bridges, alteration of the surface charge of the protein, directed evolution, and composition of consensus sequences (Lehmann and Wyss, Curr Opin Biotechnol, 12, 371-375, 2001). High thermal stability may increase the yield of the expressed protein, improve solubility or activity, decrease immunogenicity, and minimize the need of a cold chain in manufacturing. Residues that can be substituted to improve thermal stability of Tencon are residue positions 11, 14, 17, 37, 46, 73, or 86, and are described in US Pat. Publ. No. 2011/0274623. Substitutions corresponding to these residues can be incorporated to the FN3 domains or the bispecific FN3 domain containing molecules.

Measurement of protein stability and protein lability can be viewed as the same or different aspects of protein integrity. Proteins are sensitive or “labile” to denaturation caused by heat, by ultraviolet or ionizing radiation, changes in the ambient osmolarity and pH if in liquid solution, mechanical shear force imposed by small pore-size filtration, ultraviolet radiation, ionizing radiation, such as by gamma irradiation, chemical or heat dehydration, or any other action or force that may cause protein structure disruption. The stability of the molecule can be determined using standard methods. For example, the stability of a molecule can be determined by measuring the thermal melting (“TM”) temperature, the temperature in ° Celsius (° C.) at which half of the molecules become unfolded, using standard methods. Typically, the higher the TM, the more stable the molecule. In addition to heat, the chemical environment also changes the ability of the protein to maintain a particular three dimensional structure.

In one embodiment, the FN3 domains binding to the protein or nucleotide of interest exhibit increased stability by at least 5%, 10%, 15%, 20%, 25%, 30%, 35%, 40%, 45%, 50%, 55%, 60%, 65%, 70%, 75%, 80%, 85%, 90%, or 95% or more compared to the same domain prior to engineering measured by the increase in the TM.

Chemical denaturation can likewise be measured by a variety of methods. Chemical denaturants include guanidinium hydrochloride, guanidinium thiocyanate, urea, acetone, organic solvents (DMF, benzene, acetonitrile), salts (ammonium sulfate lithium bromide, lithium chloride, sodium bromide, calcium chloride, sodium chloride); reducing agents (e.g. dithiothreitol, beta-mercaptoethanol, dinitrothiobenzene, and hydrides, such as sodium borohydride), non-ionic and ionic detergents, acids (e.g. hydrochloric acid (HCl), acetic acid (CH₃COOH), halogenated acetic acids), hydrophobic molecules (e.g. phosopholipids), and targeted denaturants. Quantitation of the extent of denaturation can rely on loss of a functional property, such as ability to bind a target molecule, or by physiochemical properties, such as tendency to aggregation, exposure of formerly solvent inaccessible residues, or disruption or formation of disulfide bonds.

In one embodiment, the FN3 domains binding to the protein or nucleotide of interest exhibit increased stability by at least 5%, 10%, 15%, 20%, 25%, 30%, 35%, 40%, 45%, 50%, 55%, 60%, 65%, 70%, 75%, 80%, 85%, 90%, or 95% or more compared to the same scaffold prior to engineering measured by using guanidinium hydrochloride as a chemical denaturant. Increased stability can be measured as a function of decreased tryptophan fluorescence upon treatment with increasing concentrations of guanidine hydrochloride using well known methods.

The FN3 domains may be generated as monomers, dimers, or multimers, for example, as a means to increase the valency and thus the avidity of target molecule binding, or to generate bi- or multispecific scaffolds simultaneously binding two or more different target molecules. The dimers and multimers may be generated by linking monospecific, bi- or multispecific protein scaffolds, for example, by the inclusion of an amino acid linker, for example a linker containing poly-glycine, glycine and serine, or alanine and proline. Exemplary linker include (GS)₂, (GGGGS)₅, (AP)₂, (AP)₅, (AP)₁₀, (AP)₂₀, A(EAAAK)₅AAA, linkers. The dimers and multimers may be linked to each other in a N- to C-direction. The use of naturally occurring as well as artificial peptide linkers to connect polypeptides into novel linked fusion polypeptides is well known in the literature (Hallewell et al., J Biol Chem 264, 5260-5268, 1989; Alfthan et al., Protein Eng. 8, 725-731, 1995; Robinson & Sauer, Biochemistry 35, 109-116, 1996; U.S. Pat. No. 5,856,456). In addition, the FN3 domains may be linked to nanoparticles containing siRNA using the same or similar materials and methods as known in the art

Variants of the FN3 domain containing molecules are within the scope of the invention. For example, substitutions can be made in the FN3 domain containing molecule as long as the resulting variant retains similar selectivity and potency towards the protein or nucleotide of interest when compared to the parent molecule. Exemplary modifications are for example conservative substitutions that will result in variants with similar characteristics to those of the parent molecules. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. Alternatively, the amino acid repertoire can be grouped as (1) acidic (aspartate, glutamate); (2) basic (lysine, arginine histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (Stryer (ed.), Biochemistry, 2nd ed, WH Freeman and Co., 1981). Non-conservative substitutions can be made to the FN3 domain containing molecule that involves substitutions of amino acid residues between different classes of amino acids to improve properties of the bispecific molecules. Whether a change in the amino acid sequence of a polypeptide or fragment thereof results in a functional homolog can be readily determined by assessing the ability of the modified polypeptide or fragment to produce a response in a fashion similar to the unmodified polypeptide or fragment using the assays described herein. Peptides, polypeptides or proteins in which more than one replacement has taken place can readily be tested in the same manner.

### Half-Life Extending Moieties

The FN3 domain containing molecules may incorporate other subunits for example via covalent interaction. In one aspect of the invention, the FN3 domain containing molecules further comprise a half-life extending moiety. Exemplary half-life extending moieties are albumin, albumin-binding proteins and/or domains, transferrin or transferrin binding domains and fragments and analogues thereof, and Fc regions.

All or a portion of an antibody constant region may be attached to the molecules of the invention to impart antibody-like properties, especially those properties associated with the Fc region, such as Fc effector functions such as C1q binding, complement dependent cytotoxicity (CDC), Fc receptor binding, antibody-dependent cell-mediated cytotoxicity (ADCC), phagocytosis, down regulation of cell surface receptors (e.g., B cell receptor; BCR), and can be further modified by modifying residues in the Fc responsible for these activities (for review; see Strohl, Curr Opin Biotechnol. 20, 685-691, 2009).

Additional moieties may be incorporated into the bispecific molecules of the invention such as polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the protein scaffold coding sequences and may be generated by standard cloning and expression techniques. Alternatively, well known chemical coupling methods may be used to attach the moieties to recombinantly produced molecules of the invention.

A PEG moiety may for example be added to the FN3 domain molecules by incorporating a cysteine residue to the C-terminus of the molecule and attaching a pegyl group to the cysteine using well known methods.

### Polynucleotides, Vectors, Host Cells

The invention provides for nucleic acids encoding the FN3 domains as isolated polynucleotides or as portions of expression vectors or as portions of linear DNA sequences, including linear DNA sequences used for in vitro transcription/translation, vectors compatible with prokaryotic, eukaryotic or filamentous phage expression, secretion and/or display of the compositions or directed mutagens thereof.

In some embodiments, an isolated polynucleotide encodes the FN3 domains provided for herein. In some embodiments, the FN3 is a FN3 domain that binds to EpCAM. In some embodiments, the FN3 domain comprises the amino acid sequence of SEQ ID: 1-6, 8-11, 14-38 and 40-46 or variants thereof as described herein. In some embodiments, the FN3 domain that binds to EpCAM comprises a sequence of 14-38, or variants thereof.

The polynucleotides may be produced by chemical synthesis, such as solid phase polynucleotide synthesis on an automated polynucleotide synthesizer and assembled into complete single or double stranded molecules. Alternatively, the polynucleotides may be produced by other techniques such a PCR followed by routine cloning. Techniques for producing or obtaining polynucleotides of a given known sequence are well known in the art.

The polynucleotides may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that encode for example a marker or a tag sequence such as a histidine tag or an HA tag to facilitate purification or detection of the protein, a signal sequence, a fusion protein partner such as RepA, Fc or bacteriophage coat protein such as pIX or pIII.

Another embodiment of the invention is a vector comprising at least one polynucleotide. Such vectors may be plasmid vectors, viral vectors, vectors for baculovirus expression, transposon based vectors or any other vector suitable for introduction of the polynucleotides into a given organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

Another embodiment of the invention is a host cell comprising the vector of the invention. An FN3 domain containing molecule of the invention can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

The host cell chosen for expression may be of mammalian origin or may be selected from COS-1, COS-7, HEK293, BHK21, CHO, BSC-1, HepG2, SP2/0, HeLa, myeloma, lymphoma, yeast, insect or plant cells, or any derivative, immortalized or transformed cell thereof. Alternatively, the host cell may be selected from a species or organism incapable of glycosylating polypeptides, e.g. a prokaryotic cell or organism, such as BL21, BL21(DE3), BL21-GOLD(DE3), XL1-Blue, JM109, HMS174, HMS174(DE3), and any of the natural or engineered E. coli spp, Klebsiella spp., or Pseudomonas spp strains.

Another embodiment of the invention is a method of producing the FN3 domain containing molecule, comprising culturing the isolated host cell under conditions such that the FN3 domain containing molecule is expressed, and purifying the domain or molecule.

The FN3 domain containing molecule can be purified from recombinant cell cultures by well-known methods, for example by protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography, or high performance liquid chromatography (HPLC).

### Administration/Pharmaceutical Compositions

For therapeutic use, the FN3 domain containing molecules may be prepared as pharmaceutical compositions containing an effective amount of the domain or molecule as an active ingredient in a pharmaceutically acceptable carrier. The term “carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules of the invention in such pharmaceutical formulation can vary widely, i.e., from less than about 0.5%, usually at or at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21^{st} Edition, Troy, D. B. ed., Lipincott Williams and Wilkins, Philadelphia, Pa. 2006, Part 5, Pharmaceutical Manufacturing pp 691-1092, See especially pp. 958-989.

The mode of administration for therapeutic use of the FN3 domain containing molecules may be any suitable route that delivers the agent to the host, such as parenteral administration, e.g., intradermal, intramuscular, intraperitoneal, intravenous or subcutaneous, pulmonary; transmucosal (oral, intranasal, intravaginal, rectal); using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

Thus, a pharmaceutical composition of the invention for intramuscular injection could be prepared to contain 1 ml sterile buffered water, and between about 1 ng to about 100 mg, e.g. about 50 ng to about 30 mg or more preferably, about 5 mg to about 25 mg, of the FN3 domain of the invention. Similarly, a pharmaceutical composition of the invention for intravenous infusion could be made up to contain about 250 ml of sterile Ringer's solution, and about 1 mg to about 30 mg, e.g. about 5 mg to about 25 mg of the FN3 domain containing molecule. Actual methods for preparing parenterally administrable compositions are well known and are described in more detail in, for example, “Remington's Pharmaceutical Science”, 15th ed., Mack Publishing Company, Easton, Pa.

The FN3 domain containing molecules can be lyophilized for storage and reconstituted in a suitable carrier prior to use. This technique has been shown to be effective with conventional protein preparations and art-known lyophilization and reconstitution techniques can be employed.

The FN3 domain containing molecules may be administered to a subject in a single dose or the administration may be repeated, e.g., after one day, two days, three days, five days, six days, one week, two weeks, three weeks, one month, five weeks, six weeks, seven weeks, two months or three months. The repeated administration can be at the same dose or at a different dose. The administration can be repeated once, twice, three times, four times, five times, six times, seven times, eight times, nine times, ten times, or more.

The FN3 domain containing molecules may be administered in combination with a second therapeutic agent simultaneously, sequentially or separately. The second therapeutic agent may be a chemotherapeutic agent, an anti-angiogenic agent, or a cytotoxic drug. When used for treating cancer, the FN3 domain containing molecules may be used in combination with conventional cancer therapies, such as surgery, radiotherapy, chemotherapy or combinations thereof.

In some embodiments, a composition comprising a fibronectin type III (FN3) domain-nanoparticle complex is provided, wherein the composition comprises a FN3 domain conjugated to a surface of a nanoparticle. The conjugation can be covalent or non-covalent, such as through electrostatic interactions. In some embodiments, the FN3 domain is associated, attached, or otherwise linked to the outer surface of the nanoparticle. In some embodiments, the FN3 domain molecule is associated through the surface of the nanoparticle, similar to a transmembrane protein. In some embodiments, the FN3 domain molecule does not cross (traverse) the surface of the nanoparticle. The nanoparticles can be as provided herein, such as a lipid nanoparticle. In some embodiments, nanoparticle is Poly Lactic-co-Glycolic Acid (PLGA) nanoparticle. In some embodiments, the nanoparticle is a cyclodextrin nanoparticle, such as a cyclodextrin polymeric nanoparticle (CDP). In some embodiments, the nanoparticle is pegylated. The description of certain types of nanoparticles here is for example purposes only, and other disclosed herein or that are known can be used in the FN3 domain molecule nanoparticle complex.

In some embodiments, the nanoparticle can comprise (contain) a polynucleotide. The polynucleotide can, for example, be encapsulated by the polynucleotide. In some embodiments, the FN3 domain-nanoparticle complex comprising a polynucleotide can bind to a cell through the interaction with the FN3 domain and then deliver the polynucleotide to the cell by, for example, being internalized into the cell. The polynucleotide can be any type of polynucleotide, such as a chemically modified polynucleotide. In some embodiments, the polynucleotide is a cDNA, a siRNA, mRNA, miRNA, miRNA antagonist, dsRNA, antisense oligonucleotides (ASOs), DNA, U1 adaptor, or immunostimulatory polynucleotide, or any combination thereof. In some embodiments, the polynucleotide is a siRNA, antisense, or miRNA. In some embodiments, the polynucleotide is a siRNA or a miRNA.

In some embodiments, the nanoparticle comprises (contains) or encapsulates an active agent. The active agent can be in addition to a polynucleotide or in the place of a polynucleotide as provided for herein. In some embodiments, the active agent is a protein, peptide, small molecule compounds, or immunostimulatory agents, or any combination thereof. In some embodiments, the small molecule is a therapeutic or toxin that can be delivered to a cell type that is bound to the FN3 domain molecule.

In some embodiments, the FN3 domain binds to PSMA, EGFR, EpCam, CD22, BCMA, CD33, CD71 and/or CD8, or any combination thereof. Examples of PSMA FN3 domains can be found, for example, in U.S. application Ser. No. 15/148,312, which is hereby incorporated by reference in its entirety. Examples of EGFR FN3 domains can be found, for example, in U.S. application Ser. Nos. 14/085,340 and 14/086,250, each of which is hereby incorporated by reference in its entirety. Examples of EpCAM FN3 domains are, for example, provided for herein. Examples of EGFR FN3 domains can be found, for example, in U.S. application Ser. Nos. 14/085,340 and 14/086,250, each of which is hereby incorporated by reference in its entirety. Examples of EGFR FN3 domains can be found, for example, in U.S. application Ser. No. 15/839,915, each of which is hereby incorporated by reference in its entirety. Other FN3 domains can also be used.

As provided for herein, the FN 3 domain can be conjugated to the nanoparticle. In some embodiments, the nanoparticle is conjugated to the FN3 domain through click type cycloaddition or maleimide conjugation.

In some embodiments, the composition can also comprise a dibenzocylcooctyne (DBCO) moiety.

In some embodiments, methods of preparing a FN3 domain-nanoparticle complex are provided. In some embodiments, the methods comprise (i) panning an FN3 domain library with a protein or nucleotide of interest; (ii) recovering the FN3 domain molecule binding to the protein or nucleotide of interest; and (iii) conjugating the FN3 domain molecule with a nanoparticle.

In some embodiments, methods of preparing a FN3 domain-nanoparticle complex are provided. In some embodiments, the methods comprising contacting a FN3 domain with a nanoparticle under conditions sufficient to conjugate the FN3 domain to the nanoparticle to form the nanoparticle complex. In some embodiments, the nanoparticle comprises a polynucleotide. In some embodiments, the polynucleotide is selected from the group consisting of siRNA, mRNA, miRNA, antisense oligonucleotides (ASOs), DNA, U1 adaptor, and immunostimulatory oligonucleotide. In some embodiments, the polynucleotide is therapeutically active. In some embodiments, the complex comprises an active agent as provided for herein.

In some embodiments, a composition is provided comprising a fibronectin type III (FN3) domain conjugated to a conjugate. The conjugate can also be referred to a payload that is delivered to the cell. The delivery can be either internally through, for example, internalization of the FN3 domain into the cell, or can be external and the conjugate can interact with the cell that the FN3 domain binds to. In some embodiments, the conjugate (payload) is a provided herein. In some embodiments, the conjugate is a toxin. In some embodiments, the toxin is MMAF or MMAE. In some embodiments, the FN3 domain binds to PSMA, EGFR, EpCam, CD22, BCMA, CD33, CD71 and/or CD8. In some embodiments, the FN3 domain binds to EpCAM. In somne embodiments, the FN3 domain binding EpCAM comprises a substitution at one or more positions selected from the group consisting of Tyr25, Arg26, Pro27, Leu81, Pro82, and Tyr85. In some embodiments, the FN3 domain is a FN3 domain comprising an amino acid sequence selected from the group consisting of SEQ ID NOS: 1-6, 8-11, 14-38 and 40-46. In some embodiments, the FN3 domain comprises the amino acid sequence of SEQ ID: 1-6, 8-11, 14-38 and 40-46 or variants thereof as described herein. In some embodiments, the FN3 domain that binds to EpCAM comprises a sequence of 14-38, or variants thereof.

In some embodiments, peptides comprising a FN3 domain are provided that bind to EpCAM. In some embodiments, the FN3 domain specifically binds to EpCAM. In some embodiments, the FN3 domain comprises or consists of a sequence of SEQ ID NOS: 1-6, 8-11, 14-38 and 40-46. In some embodiments, the FN3 domain comprises the amino acid sequence of SEQ ID: 1-6, 8-11, 14-38 and 40-46 or variants thereof as described herein. In some embodiments, the FN3 domain that binds to EpCAM comprises a sequence of 14-38, or variants thereof. In some embodiments, the FN3 domain that binds EpCAM comprises an amino acid sequence that is 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to one of the amino acid sequences of SEQ ID NOS: 1-6, 8-11, 14-38 and 40-46. Percent identity can be determined using the default paramaters to align two sequences using BlastP available through the NCBI website.

In some embodiments, the sequences are as follows:

| SEQ ID NO: | Sequence |
|---|---|
| 1 | MLPAPKNLVVSRVTEDSARLSWTAPDAAFDSFQIYYSELLSYGEAIVLTVPGSE |
| | RSYDLTGLKPGTEYTVSINGVKGGTRSWSLSAIFTT |
| 2 | MLPAPKNLVVSRVTEDSARLSWTAPDAAFDSFQIYYSELLSYGEAIVLTVPGSE |
| | RSYDLTGLCPGTEYTVSINGVKGGTRSWSLSAIFTTAPAPAPAPAPLPAPKNLV |
| | VSRVTEDSARLSWTAPDAAFDSFQIYYSELLSYGEAIVLTVPGSERSYDLTGLK |
| | PGTEYTVSINGVKGGTRSWSLSAIFTTAPAPAPAPAPTIDEWLLKEAKEKAIEE |
| | LKKAGITSDYYFDLINKAKTVEGVNALKDEILKA |
| 3 | MLPAPKNLVVSRVTEDSARLSWTAPDAAFDSFQIYYSELLSYGEAIVLTVPGSE |
| | RSYDLTGLCPGTEYTVSINGVKGGTRSWSLSAIFTTAPAPAPAPAPLPAPKNLV |
| | VSRVTEDSARLSWTAPDAAFDSFQIYYSELLSYGEAIVLTVPGSERSYDLTGLC |
| | PGTEYTVSINGVKGGTRSWSLSAIFTTAPAPAPAPAPTIDEWLLKEAKEKAIEE |
| | LKKAGITSDYYFDLINKAKTVEGVNALKDEILKA |
| 4 | MLPAPKNLVVSRVTEDSARLSWTAPDAAFDSFQIYYSELLSYGEAIVLTVPGSE |
| | RSYDLTGLCPGTEYTVSINGVKGGTRSWSLSAIFTTAPCPAPAPAPLPAPKNLV |
| | VSRVTEDSARLSWTAPDAAFDSFQIYYSELLSYGEAIVLTVPGSERSYDLTGLC |
| | PGTEYTVSINGVKGGTRSWSLSAIFTTAPAPAPAPAPTIDEWLLKEAKEKAIEE |
| | LKKAGITSDYYFDLINKAKTVEGVNALKDEILKA |
| 5 | MLPAPKNLVVSRVTEDSARLSWTAPDAAFDSFQIYYSELLSYGEAIVLTVPGSE |
| | RSYDLTGLCPGTEYTVSINGVKGGTRSWSLSAIFTTAPCPAPAPAPLPAPKNLV |
| | VSRVTEDSARLSWTAPDAAFDSFQIYYSELLSYGEAIVLTVPGSERSYDLTGLC |
| | PGTEYTVSINGVKGGTRSWSLSAIFTTAPCPAPAPAPTIDEWLLKEAKEKAIEE |
| | LKKAGITSDYYFDLINKAKTVEGVNALKDEILKA |
| 6 | MLPAPKNLVVSEVTEDSARLSWTAPDAAFDSFLIQYQESEKVGEAIVLTVPGSE |
| | RSYDLTGLCPGTEYTVSIYGVKGGHRSNPLSAIFTTAPCPAPAPAPLPAPKNLV |
| | VSEVTEDSARLSWTAPDAAFDSFLIQYQESEKVGEAIVLTVPGSERSYDLTGLC |
| | PGTEYTVSIYGVKGGHRSNPLSAIFTTAPAPAPAPAPTIDEWLLKEAKEKAIEE |
| | LKKAGITSDYYFDLINKAKTVEGVNALKDEILKA |
| 8 | LPAPKNLVVSRVTEDSARLSWTAPDAAFDSFPIGYWEWDDDGEAIVLTVPGSER |
| | SYDLTGLKPGTEYHVYIAGVKGGQWSFPLSAIFTT |
| 9 | LPAPKNLVVSRVTEDSARLSWEWWVIPGDFDSFLIQYQESEKVGEAIVLTVPGS |
| | ERSYDLTGLKPGTEYTVSIYGVVNSGQWNDTSNPLSAIFTT |
| 10 | LPAPKNLVVSRVTEDSARLSWTAPDAAFDSFAIGYWEWDDDGEAIVLTVPGSER |
| | SYDLTGLKPGTEYPVYIAGVKGGQWSFPLSAIFTT |
| 11 | LPAPKNLVVSRVTEDSARLSWDIDEQRDWFDSFLIQYQESEKVGEAIVLTVPGS |
| | ERSYDLTGLKPGTEYTVSIYGVYHVYRSSNPLSAIFTT |
| 14 | MLPAPKNLVVSRVTEDSARLSWKHYRPGARFDSFLIQYQESEKVGEAIVLTVPG |
| | SERSYDLTGLKPGTEYTVSIYGVVTALPSYYSSNPLSAIFTTGGHHHHHHGGC |
| 15 | MLPAPKNLVVSRVTEDSARLSWKHYRPGARFDSFLIQYQESEKVGEAIVLTVPG |
| | SERSYDLTGLKPGTEYTVSIYGVAAHAIPRYASNPLSAIFTTGGHHHHHHGGC |
| 16 | MLPAPKNLVVSRVTEDSARLSWHNHRPQFDSFLIQYQESEKVGEAIVLTVPGSE |
| | RSYDLTGLKPGTEYTVSIYGVAIAVPWNYQSNPLSAIFTTGGHHHHHHGGC |
| 17 | MLPAPKNLVVSRVTEDSARLSWKHYRPGARFDSFLIQYQESEKVGEAIILTVPG |
| | SERSYDLTGLKPGTEYTVSIYGVVTHALPTAYTSNPLSAIFTTGGHHHHHHGGL |
| | PETGGH |
| 18 | MLPAPKNLVVSRVTEDSARLSWKHYRPGARFDSFLIQYQESEKVGEAIVLTVPG |
| | SERSYDLTGLKPGTEYTVSIYGVVAALPNNYASNPLSAIFTTGGHHHHHHGGLP |
| | ETGGH |
| 19 | MLPAPKNLVVSRVTEDSARLSWKHYRPGARFDSFLIQYQESEKVGEAIVLTVPG |
| | SERSYDLTGLKPGTEYTVSIYGVVTALPSYYSSNPLSAIFTTGGHHHHHHGGLP |
| | ETGGH |
| 20 | MLPAPKNLVVSRVTEDSARLSWKHYRPGARFDSFLIQYQESEKVGEAIVLTVPG |
| | SERSYDLTGLKPGTEYTVSIYGVVTALPSNYISNPLSAIFTTGGHHHHHHGGLP |
| | ETGGH |
| 21 | MLPAPKNLVVSRVTEDSARLSWDQYRKYAGFDSFLIQYQESEKVGEAIVLTVPG |
| | SERSYDLTGLKPGTEYTVSIYGVVTHALPQTYQSNPLSAIFTTGGHHHHHHGGL |
| | PETGGH |
| 22 | MLPAPKNLVVSRVTEDSARLSWKHYRPGARFDSFLIQYQESEKVGEAIVLTVPG |
| | SERSYDLTGLKPGTEYTVSIYGVIWGALPNSYSSNPLSAIFTTGGHHHHHHGGL |
| | PETGGH |
| 23 | MLPAPKNLVVSRVTEDSARLSWKHYRPGARFDSFLIQYQESEKVGEAIVLTVPG |
| | SERSYDLTGLKPGTEYTVSIYGVVNNALPRWYISNPLSAIFTTGGHHHHHHGGL |
| | PETGGH |
| 24 | MLPAPKNLVVSRVTEDSARLSWAHYRPGARFDSFLIQYQESEKVGEAIVLTVPG |
| | SERSYDLTGLKPGTEYTVSIYGVVTALPSYYSSNPLSAIFTTGGHHHHHHGGLP |
| | ETGGH |
| 25 | MLPAPKNLVVSRVTEDSARLSWKAYRPGARFDSFLIQYQESEKVGEAIVLTVPG |
| | SERSYDLTGLKPGTEYTVSIYGVVTALPSYYSSNPLSAIFTTGGHHHHHHGGLP |
| | ETGGH |
| 26 | MLPAPKNLVVSRVTEDSARLSWKHARPGARFDSFLIQYQESEKVGEAIVLTVPG |
| | SERSYDLTGLKPGTEYTVSIYGVVTALPSYYSSNPLSAIFTTGGHHHHHHGGLP |
| | ETGGH |
| 27 | MLPAPKNLVVSRVTEDSARLSWKHYAPGARFDSFLIQYQESEKVGEAIVLTVPG |
| | SERSYDLTGLKPGTEYTVSIYGVVTALPSYYSSNPLSAIFTTGGHHHHHHGGLP |
| | ETGGH |
| 28 | MLPAPKNLVVSRVTEDSARLSWKHYRAGARFDSFLIQYQESEKVGEAIVLTVPG |
| | SERSYDLTGLKPGTEYTVSIYGVVTALPSYYSSNPLSAIFTTGGHHHHHHGGLP |
| | ETGGH |
| 29 | MLPAPKNLVVSRVTEDSARLSWKHYRPAARFDSFLIQYQESEKVGEAIVLTVPG |
| | SERSYDLTGLKPGTEYTVSIYGVVTALPSYYSSNPLSAIFTTGGHHHHHHGGLP |
| | ETGGH |
| 30 | MLPAPKNLVVSRVTEDSARLSWKHYRPGAAFDSFLIQYQESEKVGEAIVLTVPG |
| | SERSYDLTGLKPGTEYTVSIYGVVTALPSYYSSNPLSAIFTTGGHHHHHHGGLP |
| | ETGGH |
| 31 | MLPAPKNLVVSRVTEDSARLSWKHYRPGARFDSFLIQYQESEKVGEAIVLTVPG |
| | SERSYDLTGLKPGTEYTVSIYGVATALPSYYSSNPLSAIFTTGGHHHHHHGGLP |
| | ETGGH |
| 32 | MLPAPKNLVVSRVTEDSARLSWKHYRPGARFDSFLIQYQESEKVGEAIVLTVPG |
| | SERSYDLTGLKPGTEYTVSIYGVVAALPSYYSSNPLSAIFTTGGHHHHHHGGLP |
| | ETGGH |
| 33 | MLPAPKNLVVSRVTEDSARLSWKHYRPGARFDSFLIQYQESEKVGEAIVLTVPG |
| | SERSYDLTGLKPGTEYTVSIYGVVTAAPSYYSSNPLSAIFTTGGHHHHHHGGLP |
| | ETGGH |
| 34 | MLPAPKNLVVSRVTEDSARLSWKHYRPGARFDSFLIQYQESEKVGEAIVLTVPG |
| | SERSYDLTGLKPGTEYTVSIYGVVTALASYYSSNPLSAIFTTGGHHHHHHGGLP |
| | ETGGH |
| 35 | MLPAPKNLVVSRVTEDSARLSWKHYRPGARFDSFLIQYQESEKVGEAIVLTVPG |
| | SERSYDLTGLKPGTEYTVSIYGVVTALPAYYSSNPLSAIFTTGGHHHHHHGGLP |
| | ETGGH |
| 36 | MLPAPKNLVVSRVTEDSARLSWKHYRPGARFDSFLIQYQESEKVGEAIVLTVPG |
| | SERSYDLTGLKPGTEYTVSIYGVVTALPSAYSSNPLSAIFTTGGHHHHHHGGLP |
| | ETGGH |
| 37 | MLPAPKNLVVSRVTEDSARLSWKHYRPGARFDSFLIQYQESEKVGEAIVLTVPG |
| | SERSYDLTGLKPGTEYTVSIYGVVTALPSYASSNPLSAIFTTGGHHHHHHGGLP |
| | ETGGH |
| 38 | MLPAPKNLVVSRVTEDSARLSWKHYRPGARFDSFLIQYQESEKVGEAIVLTVPG |
| | SERSYDLTGLKPGTEYTVSIYGVVTALPSYYASNPLSAIFTTGGHHHHHHGGLP |
| | ETGGH |
| 40 | MLPAPKNLVVSEVTCDSARLSWDDPWAFYESFLIQYQESEKVGEAIVLTVPGSE |
| | RSYDLTGLKPGTEYTVSIYGVHNVYKDTNMRGLPLSAIFTTGGHHHHHH |
| 41 | MLPAPKNLVVSCVTEDSARLSWDDPWAFYESFLIQYQESEKVGEAIVLTVPGSE |
| | RSYDLTGLKPGTEYTVSIYGVHNVYKDTNMRGLPLSAIFTTGGHHHHHH |
| 42 | MLPAPKNLVVSEVTEDSACLSWDDPWAFYESFLIQYQESEKVGEAIVLTVPGSE |
| | RSYDLTGLKPGTEYTVSIYGVHNVYKDTNMRGLPLSAIFTTGGHHHHHH |
| 43 | MLPAPKNLVVSEVTEDSARLSWDDPWAFYESFLIQYQESEKVGEAIVLTVPGSC |
| | RSYDLTGLKPGTEYTVSIYGVHNVYKDTNMRGLPLSAIFTTGGHHHHHH |
| 44 | MLPAPKNLVVSEVTEDSARLSWDDPWAFYESFLIQYQESEKVGEAIVLTVPGSE |
| | RSYDLTGLCPGTEYTVSIYGVHNVYKDTNMRGLPLSAIFTTGGHHHHHH |
| 45 | MLPAPKNLVVSEVTEDSARLSWDDPWAFYESFLIQYQESEKVGEAIVLTVPGSE |
| | RSYDLTGLKPGTEYTVSIYGVHNVYKDTNMRGLPLSAIFTTGGHHHHHHC |
| 46 | MLPAPKNLVVSRVTEDSARLSWTAPDAAFDSFQIYYSELLSYGEAIVLTVPGSE |
| | RSYDLTGLKPGTEYTVSINGVKGGTRSWSLSAIFTTGGHHHHHHC |

In some embodiments, the FN3 domain contains 1-5 substitutions or mutations of the sequences provided herein. In some embodiments, the protein comprises a sequence of SEQ ID NOS: 1-6, 8-11, 14-38 and 40-46, wherein at least one residue is substituted with a cysteine at a position corresponding to a residue at a position of 6, 8, 10, 11, 14, 15, 16, 20, 30, 34, 38, 40, 41, 45, 47, 48, 53, 54, 59, 60, 62, 64, 70, 88, 89, 90, 91, and 93. In some embodiments, the peptide comprises a cysteine at a position corresponding to a residue at a position of 6, 8, 10, 11, 14, 15, 16, 20, 30, 34, 38, 40, 41, 45, 47, 48, 53, 54, 59, 60, 62, 64, 70, 88, 89, 90, 91, and 93.

In some embodiments, methods of targeting a cell expressing EpCAM or other FN3 domain described herein are provided. In some embodiments, the method comprises contacting a cell with a FN3 domain that binds to EpCAM or other FN3 domain provided for herein. In some embodiments, the FN3 domain that binds to EpCAM or other FN3 domain is conjugated to a conjugate (payload). In addition to the conjugates (payloads) described herein, the conjugates can also include the FN3 domain conjugated to a heterologous molecule. In some embodiments, the heterologous molecule is a detectable label or a therapeutic agent, such as, but not limited to, a cytotoxic agent. In some embodiments, the FN3 domain that binds to EpCAM is conjugated to a detectable label. Non-limiting examples of detectable labels are provided for herein.

In some embodiments, an FN3 domain that binds EPCAM conjugated to a therapeutic agent is provided. Non-limiting examples of therapeutic agents, such as, but not limited to, cytotoxic agents, polynueltocies, or other types of therapeutics, such as, but not limited to, those that are provided for herein.

The FN3 domains that bind EPCAM conjugated to a detectable label can be used to evaluate expression of EPCAM on samples such as tumor tissue in vivo or in vitro.

Detectable labels include compositions that when conjugated to the FN3 domains that bind EPCAM renders EPCAM detectable, via spectroscopic, photochemical, biochemical, immunochemical, or other chemical methods.

Exemplary detectable labels include, but are not limited to, radioactive isotopes, magnetic beads, metallic beads, colloidal particles, fluorescent dyes, electron-dense reagents, enzymes (for example, as commonly used in an ELISA), biotin, digoxigenin, haptens, luminescent molecules, chemiluminescent molecules, fluorochromes, fluorophores, fluorescent quenching agents, colored molecules, radioactive isotopes, cintillants, avidin, streptavidin, protein A, protein G, antibodies or fragments thereof, polyhistidine, Ni²⁺, Flag tags, myc tags, heavy metals, enzymes, alkaline phosphatase, peroxidase, luciferase, electron donors/acceptors, acridinium esters, and colorimetric substrates.

A detectable label may emit a signal spontaneously, such as when the detectable label is a radioactive isotope. In some embodiments, the detectable label emits a signal as a result of being stimulated by an external stimulus, such as a magnetic or electric, or electromagentic field.

Exemplary radioactive isotopes may be γ-emitting, Auger-emitting, β-emitting, an alpha-emitting or positron-emitting radioactive isotope. Exemplary radioactive isotopes include ³H, ¹¹C, ¹⁵N, ¹⁸F, ¹⁹F, ⁵⁵Co, ⁵⁷Co, ⁶⁰Co, ⁶¹Cu, ⁶²Cu, ⁶⁴Cu, ⁶⁷Cu, ⁶⁸Ga, ⁷²As, ⁷⁵Br, ⁸⁶Y, ⁸⁹Zr, ⁹⁰Sr, ^{94m}Tc, ^{99m}Tc, ¹¹⁵In, ¹²³I, ¹²⁴I, ¹²⁵I, ¹³¹I, ²¹¹At, ²¹²Bi, ²¹³Bi, ²²³Ra, ²²⁶Ra, ²²⁵Ac, and ²²⁷Ac.

Exemplary metal atoms are metals with an atomic number greater than 20, such as calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms.

In some embodiments, the metal atoms may be alkaline earth metals with an atomic number greater than twenty.

In some embodiments, the metal atoms may be lanthanides.

In some embodiments, the metal atoms may be actinides.

In some embodiments, the metal atoms may be transition metals.

In some embodiments, the metal atoms may be poor metals.

In some embodiments, the metal atoms may be gold atoms, bismuth atoms, tantalum atoms, and gadolinium atoms.

In some embodiments, the metal atoms may be metals with an atomic number of 53 (i.e., iodine) to 83 (i.e., bismuth).

In some embodiments, the metal atoms may be atoms suitable for magnetic resonance imaging.

The metal atoms may be metal ions in the form of +1, +2, or +3 oxidation states, such as Ba²⁺, Bi³⁺, Cs⁺, Ca²⁺, Cr²⁺, Cr³⁺, Cr⁶⁺, Co²⁺, Co³⁺, Cu⁺, Cu²⁺, Cu³⁺, Ga³⁺, Gd³⁺+, Au⁺, Au³⁺, Fe²⁺, Fe³⁺, Pb²⁺, Mn²⁺, Mn³⁺, Mn⁴⁺, Mn⁷⁺, Hg²⁺, Ni²⁺, Ni³⁺, Ag⁺, Sr²⁺, Sn²⁺, Sn⁴⁺, Sn⁴⁺, and Zn²⁺. The metal atoms may comprise a metal oxide, such as iron oxide, manganese oxide, or gadolinium oxide.

Suitable dyes include any commercially available dyes such as, for example, 5(6)-carboxyfluorescein, IRDye 680RD maleimide or IRDye 800CW, ruthenium polypyridyl dyes, and the like.

Suitable fluorophores are fluorescein isothiocyante (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes.

The FN3 domains that bind EPCAM conjugated to a detectable label may be used, for example, as an imaging agent to evaluate tumor distribution, diagnosis for the presence of tumor cells and/or, recurrence of tumor.

In some embodiments, the FN3 domains that bind EPCAM are conjugated to a therapeutic agent, such as, but not limited to, a cytotoxic agent.

In some embodiments, the therapeutic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

The FN3 domains that bind EPCAM conjugated to a therapeutic agent disclosed herein may be used in the targeted delivery of the therapeutic agent to EPCAM expressing cells (e.g. tumor cells), and intracellular accumulation therein. Although not bound to any particular theory, this type of delivery can be helpful where systemic administration of these unconjugated agents may result in unacceptable levels of toxicity to normal cells.

In some embodiments, the therapeutic agent can elicit their cytotoxic and/or cytostatic effects by mechanisms such as, but not limited to, tubulin binding, DNA binding, topoisomerase inhibition, DNA cross linking, chelation, spliceosome inhibition, NAMPT inhibition, and HDAC inhibition.

In some embodiments, the therapeutic agent is a spliceosome inhibitor, a NAMPT inhibitor, or a HDAC inhibitor. In some embodiments, the agent is an immune system agonist, for example, TLR7,8,9, (dsRNA), and STING (CpG) agonists. In some embodiments, the agent is daunomycin, doxorubicin, methotrexate, vindesine, bacterial toxins such as diphtheria toxin, ricin, geldanamycin, maytansinoids or calicheamicin.

In some embodiments, the therapeutic agent is an enzymatically active toxin such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, or the tricothecenes.

In some embodiments, the therapeutic agent is a radionuclide, such as ²¹²Bi, ¹³¹I, ¹³¹In, ⁹⁰Y, or ¹⁸⁶Re.

In some embodiments, the therapeutic agent is dolastatin or dolostatin peptidic analogs and derivatives, auristatin or monomethyl auristatin phenylalanine. Exemplary molecules are disclosed in U.S. Pat. Nos. 5,635,483 and 5,780,588. Dolastatins and auristatins have been shown to interfere with microtubule dynamics, GTP hydrolysis, and nuclear and cellular division (Woyke et al (2001) Antimicrob Agents and Chemother. 45(12):3580-3584) and have anticancerand antifungal activity. The dolastatin or auristatin drug moiety may be attached to the FN3 domain through the N (amino) terminus or the C (carboxyl) terminus of the peptidic drug moiety (WO 02/088172), or via any cysteine engineered into the FN3 domain.

In some embodiments, therapeutic agent can be, for example, auristatins, camptothecins, duocarmycins, etoposides, maytansines and maytansinoids, taxanes, benzodiazepines or benzodiazepine containing drugs (e.g., pyrrolo[1,4]-benzodiazepines (PBDs), indolinobenzodiazepines, and oxazolidinobenzodiazepines) or vinca alkaloids.

In some embodiments, the FN3 domains that bind EPCAM are conjugated to a therapeutic compound, which can, for example, be used for the treatment of a cancer, autoimmune diseases of the gut, lung diseases, and the like. Examples of auto-immune disease include, but are not limited to—immune hepatitis, primary sclerosing cholangitis, Type 1 diabetes, a transplant, or GVHD, the method comprising administering a therapeutic compound of any of claim 1 to the subject to treat the auto-immune hepatitis, primary sclerosing cholangitis, Type 1 diabetes, a transplant, or GVHD. In some embodiments, the auto-immune disease includes, but is not limited to inflammatory bowel disease, Crohn's disease, ulcertiave colitis. Other examples of auto-immune diseases, include, but are not limited to, Type 1 Diabetes, Multiple Sclerosis, Cardiomyositis, vitiligo, alopecia, inflammatory bowel disease (IBD, e.g. Crohn's disease or ulcerative colitis), Sjogren's syndrome, focal segmented glomerular sclerosis (FSGS), scleroderma/systemic sclerosis (SSc) or rheumatoid arthritis, and the like.

In some embodiments, the cancer or tumor is breast cancer, lung cancer, colon cancer, or ovarian cancer. In some embodiments, the cancer is an epithelial cancer.

“Treat” or “treatment” refers to the therapeutic treatment and prophylactic measures, wherein the object is to prevent or slow down (lessen) an undesired physiological change or disorder, such as the development or spread of cancer. In some embodiments, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “therapeutically effective amount” refers to an amount effective, at dosages and for periods of time necessary, to achieve a desired therapeutic result. A therapeutically effective amount of the FN3 domains that bind EpCAM or other proteins described herein may vary according to factors such as the disease state, age, sex, and weight of the individual. Exemplary indicators of an effective FN3 domain that binds EpCAM or other protein described herein is improved well-being of the patient, decrease or shrinkage of the size of a tumor, arrested or slowed growth of a tumor, and/or absence of metastasis of cancer cells to other locations in the body.

In some embodiments, the compound for the treatment of diseases or conditions provided herein are nucleic acid molecules, such as, but not limited to, oligonucleotides, RNA interference molecules, or antisense constructs. In some embodiments, the RNA interference molecules are small interfering RNA molecules or short hairpin RNA interference molecules. In some embodiments, the RNA interference molecules are antiviral agents, for example, by interfering with the ability of a virus to replicate itself in a host, or other polynucleotides that are described herein.

The FN3 domains that specifically bind EPCAM may be conjugated to a detectable label using known methods. In some embodiments, the detectable label is complexed with a chelating agent. In some embodiments, the detectable label is conjugated to the FN3 domain that binds EPCAM via a linker.

The detectable label, therapeutic compound, or the cytotoxic compound may be linked directly, or indirectly, to the FN3 domain that binds EPCAM using known methods.

Suitable linkers are known in the art and include, for example, prosthetic groups, non-phenolic linkers (derivatives of N-succimidyl-benzoates; dodecaborate), chelating moieties of both macrocyclics and acyclic chelators, such as derivatives of 1,4,7,10-tetraazacyclododecane-1,4,7,10,tetraacetic acid (DOTA), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA), N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene) and other chelating moieties. Suitable peptide linkers are well known.

In some embodiment, the FN3 domain that binds EPCAM is removed from the blood via renal clearance.

As provided herein, in some embodiments, the conjugate (payload) is linked to a surface of the nanoparticle. The nanoparticle can be any nanoparticle as described herein for use in any of the methods provided for herein. In some embodiments, the conjugate is a complex that targets protein degradation. In some embodiments, the conjugate is a binding moiety for an E3 ubiquitin ligase. In some embodiments, the binding moiety is a PROTAC domain binds an E3 ubiquitin ligase and a target protein joined by a linker).

In some embodiments, the cell that is targeted is a cell that expresses EpCAM. In some embodiments, the cell is a breast cell, a lung cell, a colon cell, an ovarian cell. In some embodiments, the cell is an epithelial cell.

In some embodiments, methods of treating cancer in a patient are provided, the method comprising administering a composition or peptide as described herein to the patient to treat the cancer. In some embodiments, the cancer is breast cancer, lung cancer, colon cancer, or ovarian cancer. In some embodiments, the cancer is an epithelial cancer. In some embodiments, the cancer is a cancer that expresses or overexpresses EpCAM or other FN3 domain described herein.

In some embodiments, a kit comprising the FN3 domain that bind EpCAM or the complexes provided for herein are provided. The kit may be used for therapeutic uses and as a diagnostic kit.

In some embodiments, the kit comprises the FN3 domain that binds EpCAM or other proteins described herein and reagents for detecting the FN3 domain or delivering the complex or FN3 domain to the cell targeted by the FN3 domain. The kit can include one or more other elements including: instructions for use; other reagents, e.g., a label, an agent useful for chelating, or otherwise coupling, a radioprotective composition; devices or other materials for preparing the FN3 domain that binds EpCAM for administration for imaging, diagnostic or therapeutic purpose; pharmaceutically acceptable carriers; and devices or other materials for administration to a subject.

In some embodiments, the kit comprises the FN3 domain that comprising the amino acid sequences of one of SEQ ID NOS: 1-6, 8-11, 14-38 and 40-46. In some embodiments, the FN3 domain comprises the amino acid sequence of SEQ ID: 1-6, 8-11, 14-38 and 40-46 or variants thereof as described herein. In some embodiments, the FN3 domain that binds to EpCAM comprises a sequence of 14-38, or variants thereof.

In some embodiments, the FN3 domains used in the compositions or methods provided herein comprise an amino acid sequence of SEQ ID NOS: 1-6, 8-11, 14-38 and 40-46, or variants. In some embodiments, the FN3 domain that binds to EpCAM comprises a sequence of 14-38, or variants thereof.

The sequences provided herein may include a HIS tag or HIS-C tag at the N- or C-terminus of the protein. These N- or C-terminal sequences can be removed and not included. For example, SEQ ID NO: 40 is illustrated as MLPAPKNLVVSEVTCDSARLSWDDPWAFYESFLIQYQESEKVGEAIVLTVPGSERS YDLTGLKPGTEYTVSIYGVHNVYKDTNMRGLPLSAIFTTGGHGHHHHH and in some embodiments, the 6-His tag is removed to provide MLPAPKNLVVSEVTCDSARLSWDDPWAFYESFLIQYQESEKVGEAIVLTVPGSERS YDLTGLKPGTEYTVSIYGVHNVYKDTNMRGLPLSAIFTTGG (SEQ ID NO:49). The C-terminal tag cannot be included or can be used for purification or detection purposes.

While having described embodiments in certain terms, the embodiments also include the following examples, which should not be construed as limiting the scope of the claims.

### Example 1. Construction of Tencon Libraries

Tencon is an immunoglobulin-like scaffold, fibronectin type III (FN3) domain, designed from a consensus sequence of fifteen FN3 domains from human tenascin-C (Jacobs et al., Protein Engineering, Design, and Selection, 25:107-117, 2012). The crystal structure of Tencon shows six surface-exposed loops that connect seven beta-strands. These loops, or selected residues within each loop, can be randomized in order to construct libraries of fibronectin type III (FN3) domains that can be used to select novel molecules that bind to specific targets.

Tencon:
(SEQ ID NO: 39)
LPAPKNLVVSEVTEDSLRLSWTAPDAAFDSFLIQYQESEKVGEAINLTVP
GSERSYDLTGLKPGTEYTVSIYGVKGGHRSNPLSAEFTT:

### Construction of TCL1 Library

A library designed to randomize only the FG loop of Tencon, TCL1, was constructed for use with the cis-display system (Jacobs et al., Protein Engineering, Design, and Selection, 25:107-117, 2012). In this system, a single-strand DNA incorporating sequences for a Tac promoter, Tencon library coding sequence, RepA coding sequence, cis-element, and ori element is produced. Upon expression in an in vitro transcription/translation system, a complex is produced of the Tencon-RepA fusion protein bound in cis to the DNA from which it is encoded. Complexes that bind to a target molecule are then isolated and amplified by polymerase chain reaction (PCR), as described below.

Construction of the TCL1 library for use with cis-display was achieved by successive rounds of PCR to produce the final linear, double-stranded DNA molecules in two halves; the 5′ fragment contains the promoter and Tencon sequences, while the 3′ fragment contains the repA gene and the cis- and ori elements. These two halves are combined by restriction digest in order to produce the entire construct. The TCL1 library was designed to incorporate random amino acids only in the FG loop of Tencon, KGGHRSN (SEQ ID NO:50). NNS codons were used in the construction of this library, resulting in the possible incorporation of all 20 amino acids and one STOP codon into the FG loop. The TCL1 library contains six separate sub-libraries, each having a different randomized FG loop length, from 7 to 12 residues, in order to further increase diversity. Design of tencon-based libraries are shown in Table 2.

TABLE 2
BC Loop

| Library | Design | FG Loop Design |
|---|---|---|
| WT Tencon | TAPDAAFD* | KGGHRSN** |
| TCL1 | TAPDAAFD* | XXXXXXX |
| | | XXXXXXXX |
| | | XXXXXXXXX |
| | | XXXXXXXXXX |
| | | XXXXXXXXXXX |
| | | XXXXXXXXXXXX |
| TCL2 | ######## | #####S## |

*TAPDAAFD (SEQ ID NO: 51): residues 22-28;
**KGGHRSN (SEQ ID NO: 50):
X refers to degenerate amino acids encoded by NNS codons.
#refers to the “designed distribution of amino acids”
described in the text.

To construct the TCL1 library, successive rounds of PCR were performed to append the Tac promoter, build degeneracy into the F:G loop, and add necessary restriction sites for final assembly. First, a DNA sequence containing the promoter sequence and Tencon sequence 5′ of the FG loop was generated by PCR in two steps. DNA corresponding to the full Tencon gene sequence was used as a PCR template with primers POP2220 and TC5′ to FG. The resulting PCR product from this reaction was used as a template for the next round of PCR amplification with primers 130mer and Tc5′toFG to complete the appending of the 5′ and promoter sequences to Tencon. Next, diversity was introduced into the F:G loop by amplifying the DNA product produced in the first step with forward primer POP2222, and reverse primers TCF7, TCF8, TCF9, TCF10, TCF11, or TCF12, which contain degenerate nucleotides. At least eight 100 μL PCR reactions were performed for each sub-library to minimize PCR cycles and maximize the diversity of the library. At least 5 μg of this PCR product were gel-purified and used in a subsequent PCR step, with primers POP2222 and POP2234, resulting in the attachment of a 6×His tag and NotI restriction site to the 3′ end of the Tencon sequence. This PCR reaction was carried out using only fifteen PCR cycles and at least 500 ng of template DNA. The resulting PCR product was gel-purified, digested with NotI restriction enzyme, and purified by Qiagen column.

The 3′ fragment of the library is a constant DNA sequence containing elements for display, including a PspOMI restriction site, the coding region of the repA gene, and the cis- and ori elements. PCR reactions were performed using a plasmid (pCR4Blunt) (Invitrogen) containing this DNA fragment with M13 Forward and M13 Reverse primers. The resulting PCR products were digested by PspOMI overnight and gel-purified. To ligate the 5′ portion of library DNA to the 3′ DNA containing the repA gene, 2 pmol of 5′ DNA were ligated to an equal molar amount of 3′ repA DNA in the presence of NotI and PspOMI enzymes and T4 ligase. After overnight ligation at 37° C., a small portion of the ligated DNA was run on a gel to check ligation efficiency. The ligated library product was split into twelve PCR amplifications and a 12-cycle PCR reaction was run with primer pair POP2250 and DigLigRev. The DNA yield for each sub-library of TCL1 library ranged from 32-34 μg.

To assess the quality of the library, a small portion of the working library was amplified with primers Tcon5new2 and Tcon6, and was cloned into a modified pET vector via ligase-independent cloning. The plasmid DNA was transformed into BL21-GOLD (DE3) competent cells (Stratagene) and 96 randomly picked colonies were sequenced using a T7 promoter primer. No duplicate sequences were found. Overall, approximately 70-85% of clones had a complete promoter and Tencon coding sequence without frame-shift mutation. The functional sequence rate, which excludes clones with STOP codons, was between 59% and 80%.

### Construction of TCL2 Library

TCL2 library was constructed in which both the BC and FG loops of Tencon were randomized and the distribution of amino acids at each position was strictly controlled. Table 3 shows the amino acid distribution at desired loop positions in the TCL2 library. The designed amino acid distribution had two aims. First, the library was biased toward residues that were predicted to be structurally important for Tencon folding and stability based on analysis of the Tencon crystal structure and/or from homology modeling. For example, position 29 was fixed to be only a subset of hydrophobic amino acids, as this residue was buried in the hydrophobic core of the Tencon fold. A second layer of design included biasing the amino acid distribution toward that of residues preferentially found in the heavy chain HCDR3 of antibodies, to efficiently produce high-affinity binders (Birtalan et al., J Mol Biol 377:1518-28, 2008; Olson et al., Protein Sci 16:476-84, 2007). Towards this goal, the “designed distribution” of Table 3 refers to the distribution as follows: 6% alanine, 6% arginine, 3.9% asparagine, 7.5% aspartic acid, 2.5% glutamic acid, 1.5% glutamine, 15% glycine, 2.3% histidine, 2.5% isoleucine, 5% leucine, 1.5% lysine, 2.5% phenylalanine, 4% proline, 10% serine, 4.5% threonine, 4% tryptophan, 17.3% tyrosine, and 4% valine. This distribution is devoid of methionine, cysteine, and STOP codons.

TABLE 3

| Residue | | |
|---|---|---|
| Position* | WT residues | Distribution in the TCL2 library |
| 22 | T | designed distribution |
| 23 | A | designed distribution |
| 24 | P | 50% P + designed distribution |
| 25 | D | designed distribution |
| 26 | A | 20% A + 20% G + designed distribution |
| 27 | A | designed distribution |
| 28 | F | 20% F, 20% I, 20% L, 20% V, 20% Y |
| 29 | D | 33% D, 33% E, 33% T |
| 75 | K | designed distribution |
| 76 | G | designed distribution |
| 77 | G | designed distribution |
| 78 | H | designed distribution |
| 79 | R | designed distribution |
| 80 | S | 100% S |
| 81 | N | designed distribution |
| 82 | P | 50% P + designed distribution |

*residue numbering is based on Tencon sequence

The 5′ fragment of the TCL2 library contained the promoter and the coding region of Tencon, which was chemically synthesized as a library pool (Sloning Biotechnology). This pool of DNA contained at least 1×10¹¹ different members. At the end of the fragment, a BsaI restriction site was included in the design for ligation to RepA.

The 3′ fragment of the library was a constant DNA sequence containing elements for display including a 6×His tag, the coding region of the repA gene, and the cis-element. The DNA was prepared by PCR reaction using an existing DNA template (above), and primers LS1008 and DigLigRev. To assemble the complete TCL2 library, a total of 1 μg of BsaI-digested 5′ Tencon library DNA was ligated to 3.5 μg of the 3′ fragment that was prepared by restriction digestion with the same enzyme. After overnight ligation, the DNA was purified by Qiagen column and the DNA was quantified by measuring absorbance at 260 nm. The ligated library product was amplified by a 12-cycle PCR reaction with primer pair POP2250 and DigLigRev. A total of 72 reactions were performed, each containing 50 ng of ligated DNA products as a template. The total yield of TCL2 working library DNA was about 100 μg. A small portion of the working library was sub-cloned and sequenced, as described above for library TCL1. No duplicate sequences were found. About 80% of the sequences contained complete promoter and Tencon coding sequences with no frame-shift mutations.

### Construction of TCL14 Library

The top (BC, DE, and FG) and the bottom (AB, CD, and EF) loops, e.g., the reported binding surfaces in the FN3 domains are separated by the beta-strands that form the center of the FN3 structure. Alternative surfaces residing on the two “sides” of the FN3 domains having different shapes than the surfaces formed by loops only are formed at one side of the FN3 domain by two anti-parallel beta-strands, the C and the F beta-strands, and the CD and FG loops, and is herein called the C-CD-F-FG surface.

A library randomizing an alternative surface of Tencon was generated by randomizing select surface exposed residues of the C and F strands, as well as portions of the CD and FG loops. A Tencon variant, Tencon27 having following substitutions when compared to Tencon was used to generate the library; E11R L17A, N46V, E86I. A full description of the methods used to construct this library is described in U.S. patent application Ser. No. 13/852,930.

### Example 2: Selection of Fibronectin Type III (FN3) Domains that Bind a Cellular Target

### Library Screening

Various methods can be used to pan any of the FN3 domain libraries described herein to obtain FN3 domains that bind to a protein or nucleotide of interest for targeting use in the invention. For example, cis-display can be used to select FN3 domains from the TCL1 and TCL2 libraries. A recombinant human protein, possibly fused to an IgG1 Fc, can be used with standard methods for panning.

### Selection of Anti-hEGFR FN3 Domain Molecule G3

Cis-display was used to select EGFR binding FN3 domain molecules as described in U.S. patent application Ser. No. 13/852,930. Briefly, recombinant human EGFR-ECD encompassing residues 25-645 fused to the Fc domain of human IgG₁ was purchased from R&D Systems and biotinylated for selections. For in vitro transcription and translation (ITT), 2-3 μg of TCL14 DNA was incubated with 0.1 mM complete amino acids, 1×S30 premix components, and 15 μL of S30 extract (Promega) in a total volume of 50 μL and incubated at 30° C. After 1 hour, 450 μL of blocking solution (PBS pH 7.4, supplemented with 2% bovine serum albumin, 100 μg/mL herring sperm DNA, and 1 mg/mL heparin) were added and the reaction allowed to incubate on ice for 15 minutes. EGFR-Fc:EGF complexes were assembled at molar ratios of 1:1 EGFR to EGF by mixing recombinant human EGF (R&D Systems) with biotinylated recombinant EGFR-Fc in blocking solution for 1 hour at room temperature. For binding, 500 μL of blocked ITT reactions were mixed with 100 μL of EGFR-Fc:EGF complexes and incubated for 1 hour at room temperature, after which bound complexes were pulled down with magnetic neutravidin or streptavidin beads (Seradyne). Unbound library members were removed by successive washes with PBST and PBS. After washing, DNA was eluted from the bound complexes by heating to 75° C. for 10 minutes, amplified by PCR, and attached to a DNA fragment encoding RepA by restriction digestion and ligation for further rounds of panning. High affinity binders were isolated by successively lowering the concentration of target EGFR-Fc during each round from 500 nM to 2.5 nM and increasing the washing stringency. In rounds 6 & 7, unbound and weakly bound Centyrins were removed by washing in the presence of a 200-fold molar excess of non-biotinylated EGFR-Fc for 1 hour in PBST. In rounds 8 & 9, unbound and weakly bound Centyrins were removed by washing in the presence of a 2000-fold molar excess of non-biotinylated EGFR-Fc for 1 hour in PBST. FN3 domains were cloned into an expression vector and screened for binding to hEGFR as described.

### Example 3: Engineering of FN3 Domains

The FN3 domains can be engineered to increase the conformational stability of each molecule. The mutations L17A, N46V, and E86I (described in US Pat. Publ. No. 2011/0274623) can be incorporated into the molecules by DNA synthesis. Differential scanning calorimetry in PBS can be used to assess the stability of each mutant in order to compare it to that of the corresponding parent molecule.

### Example 4: Cysteine Engineering of FN3 Domains

Cysteine mutants of FN3 domains are made from the base Tencon molecule and variants thereof that do not have cysteine residues. These mutations may be made using standard molecular biology techniques known in the art to incorporate a unique cysteine residue into the base Tencon sequence or other FN3 domains in order to serve as a site for chemical conjugation of small molecule drugs, fluorescent tags, polyethylene glycol, or any number of other chemical entities. The site of mutation to be selected should meet certain criteria. For example, the Tencon molecule mutated to contain the free cysteine should: (i) be highly expressed in E. coli, (ii) maintain a high level of solubility and thermal stability, and (iii) maintain binding to the target antigen upon conjugation. Since the Tencon scaffold is only ˜90-95 amino acids, single-cysteine variants can easily be constructed at every position of the scaffold to rigorously determine the ideal position(s) for chemical conjugation.

### Example 5: Targeting Nanoparticles with FN3 Domain Molecules Via Unique Cysteine Placement

To establish the utility of FN3 domains in targeted nanomedicine, experiments were performed to show the advantages provided by FN3 domain molecules for target engagement and nanoparticle-FN3 domain coupling reactions. For initial conjugation and targeting experiments, 6 variants of an EGFR-targeting FN3 domain (P54AR4-83v2) were created in which a single unique cysteine was placed in different locations within the protein for coupling with nanoparticles as described in U.S. patent application Ser. No. 14/512,801. These proteins were named 83v10, 83v11, 83v12, 83v13, 83v14 and 83v15. The amino acid sequence for each of these proteins is as follows:

83v10 = E15C
(SEQ ID No: 40)
MLPAPKNLVVSEVTCDSARLSWDDPWAFYESFLIQYQESEKVGEAIVLTV
PGSERSYDLTGLKPGTEYTVSIYGVHNVYKDTNMRGLPLSAIFTTGGHHH
HHH
83v11 = E12C
(SEQ ID No: 41)
MLPAPKNLVVSCVTEDSARLSWDDPWAFYESFLIQYQESEKVGEAIVLTV
PGSERSYDLTGLKPGTEYTVSIYGVHNVYKDTNMRGLPLSAIFTTGGHHH
HHH
83v12 = R19C
(SEQ ID No: 42)
MLPAPKNLVVSEVTEDSACLSWDDPWAFYESFLIQYQESEKVGEAIVLTV
PGSERSYDLTGLKPGTEYTVSIYGVHNVYKDTNMRGLPLSAIFTTGGHHH
HHH
83v13 = E54C
(SEQ ID No: 43)
MLPAPKNLVVSEVTEDSARLSWDDPWAFYESFLIQYQESEKVGEAIVLTV
PGSCRSYDLTGLKPGTEYTVSIYGVHNVYKDTNMRGLPLSAIFTTGGHHH
HHH
83v14 = K63C
(SEQ ID No: 44)
MLPAPKNLVVSEVTEDSARLSWDDPWAFYESFLIQYQESEKVGEAIVLTV
PGSERSYDLTGLCPGTEYTVSIYGVHNVYKDTNMRGLPLSAIFTTGGHHH
HHH
83v15 = C-terminal
(SEQ ID No: 45)
MLPAPKNLVVSEVTEDSARLSWDDPWAFYESFLIQYQESEKVGEAIVLTV
PGSERSYDLTGLKPGTEYTVSIYGVHNVYKDTNMRGLPLSAIFTTGGHHH
HHHC
G3= C-terminal
(SEQ ID No: 46)
MLPAPKNLVVSRVTEDSARLSWTAPDAAFDSFQIYYSELLSYGEAIVLTV
PGSERSYDLTGLKPGTEYTVSINGVKGGTRSWSLSAIFTTGGHHHHHHC

### Conjugation

Superparamagnetic iron oxide nanoparticles (SPION) were synthesized and surface functionalized with amine chemical handles for downstream conjugation with targeting elements. From this nanoparticle stock, 50 μL of 1 mg Fe/mL SPION was aliquotted into six separate eppendorf tubes. Each FN3 domain molecule was added to the SPION sample so that a final 50:1 molar ratio of FN3 domain molecule: SPION was achieved. Samples were allowed to react for 4 hours at RT on a thermomixer, 1250 rpm. Samples were purified following the 4 hours via magnetic purification on MACS LS columns. Samples were then concentrated with a single 5 minute spin (5500×rcf) using microcon 30 kDa columns from millipore.

Shortly after setting up the above conjugation reactions, it was noted that a couple of the conjugate samples had precipitated. Specifically, 83v11 and 83v14 precipitated. This left three samples, 83v10, 83v12 and 83v15 as potential stable conjugates.

For the remaining soluble samples, an iron concentration assay was performed. Specifically, 10 μL of SPION samples were incubated with 10 μL of 3% H₂O₂ and 1 mL of 6M HCl for 10 minutes in the dark. Samples were then assessed for their absorbance at 410 nm. Absorbance measurements were compared to standard curves for dissolved iron to determine mg/mL concentration. Further, assuming an average iron core size of 5 nm and roughly spherical particles, the mg/mL concentration of iron was converted to a molar concentration using the 113,008 g/mole as the estimated molecular weight of the SPIO nanoparticles. Concentrations were calculated as shown in Table 4:

TABLE 4
Concentrations of Centryin-SPIO nanoparticles.

| | Absorbance at | Conc. Fe | Molar |
|---|---|---|---|
| Sample | 410 nm | (mg/mL) | (μM) |
| TenCon25-SPION | 0.08 | 0.336 | 2.97 |
| 83v10-SPION | 0.106 | 0.445 | 3.94 |
| 83v12-SPION | 0.156 | 0.656 | 5.80 |
| 83v15-SPION | 0.153 | 0.643 | 5.69 |

### Cell Binding Assays

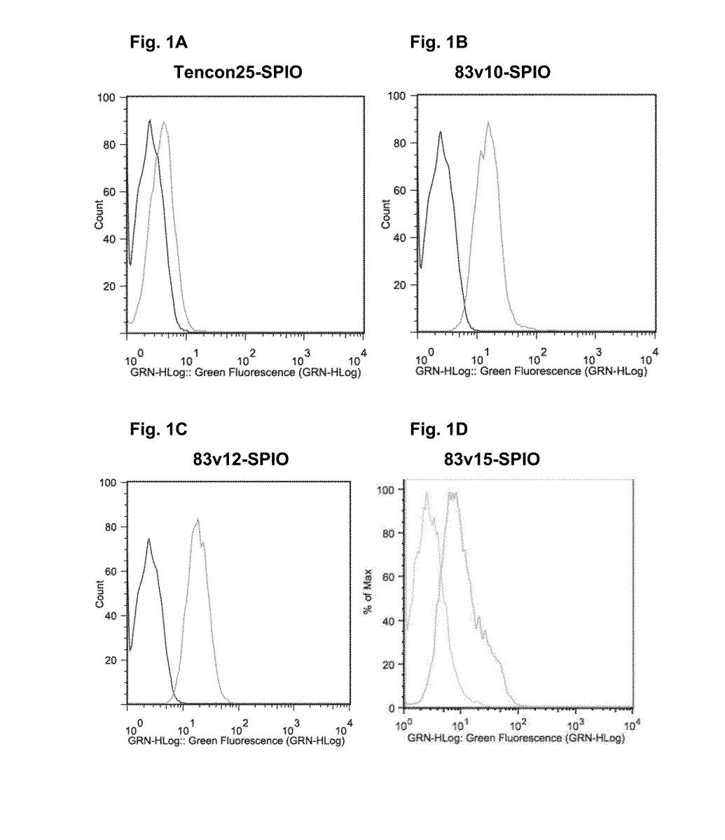

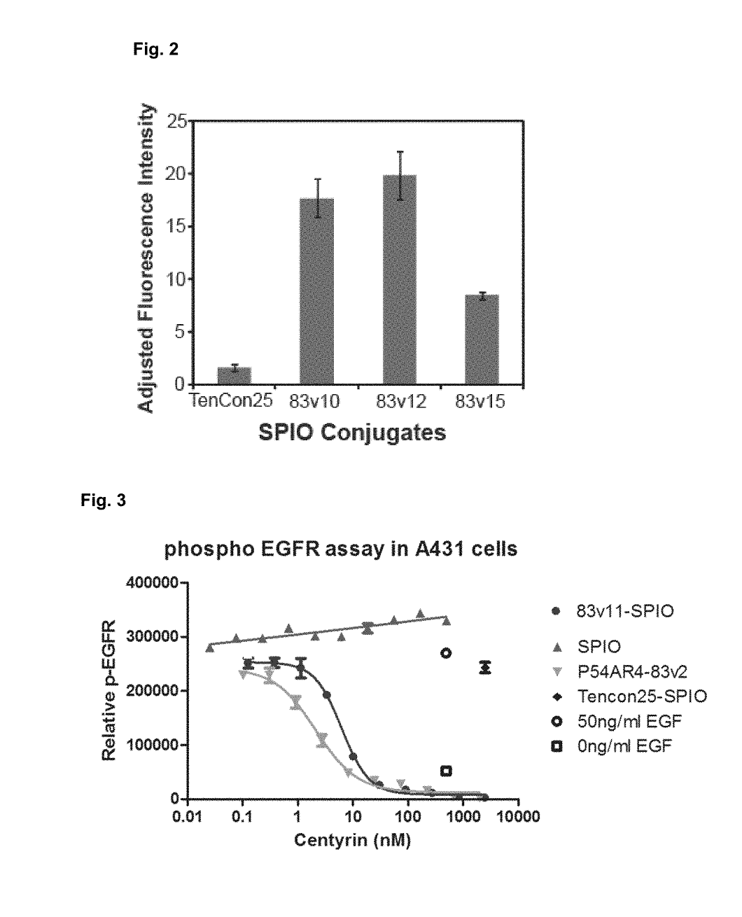

To determine the activity of the recently created FN3 domain molecule-SPION, cell targeting assays were set-up in vitro. H292 cells were dissociated from a T-150 cell culture flask using Hank's Enzyme free dissociation buffer (Invitrogen—13150-016). The cells and dissociation buffer were incubated for 15 minutes in a cell incubator. The flask was gently tapped to dissociate any residually bound cells and the buffer and cells were transferred to a 15 mL conical tube. The cells were spun at 1,000 rcf for 2 minutes. The supernatant was gently removed with careful consideration paid to not disturbing the pelleted cells. The cell pellet left over was reconstituted in 1.5 mL of phenol-red free RPMI 1640 media with 10% FBS. It was determined that the suspension contained approximately 1.5×10¹\6 cells. 100 μL of this suspension was added to each well of a 96-well plate. FN3 domain molecule-SPION sample concentrations are listed above. From these known concentrations, FN3 domain molecule-SPION was added to each well so that the final concentration of FN3 domain molecule-SPION with the cells was 100 μg/mL Fe. Samples were plated in triplicate and incubated for 30 minutes and then purified via 3× centrifugation at 1,000×rcf and resuspension in dPBS pH 7.2. Samples were suspended in a final volume of 300 μL and moved to a clear, flat bottom 96-well plate for analysis on the Guava HTS flow cytometer. A total of 8,000 cells were counted for each sample. Following flow, population samples were analyzed using FlowJo. Each sample was gated so that only live cells were assessed for fluorescence. Histograms were obtained wherein the black peak (the peak to the left side of the graph) is the autofluorescence obtained from unlabeled H292 cells. The blue peak (the peak to the right side of the graph) corresponds to the sample title above the histogram. 83v10, 83v12 and 83v15 are positively shifted compared to the tencon25 control or blank cells (FIGS. 1A-1D). A bar graph shows a single quantitative comparison of all samples (FIG. 2). All samples were normalized by subtracting the blank cell autofluorescence from the mean fluorescent intensities of each sample. Statistical significance (p<0.005) was found for 83v10-SPION, 83v12-SPION and 83v15-SPION when compared to the TenCon25-SPION sample.

### FN3 Domain Molecule-Nanoparticle Cell Binding Assessment

In addition to target specific binding, some FN3 domain molecules were selected to block a biological pathway by inhibiting ligand binding. To assess whether FN3 domain molecule activity is retained following conjugation to nanoparticles, activity assessments were set-up for EGFR-targeted nanoparticles using the following protocol and set-up:

### Cell Preparation

A431 cells were plated at 20k per well in a 96 well plate. Following a 24-hour incubation, plate media was switched to serum free RPMI+GlutaMAX. Cells were incubated overnight. On the following day, cell playes were blocked using Blocker A buffer for 1 hour at RT shaking at setting 2. Cells were then starved for 20-24 hours and cell media was removed from cells by aspiration.

FN3 domain molecule-SPION Cell Treatment

Inhibitors (83v11-SPION, Blank SPION and 83v2) were diluted in serum-free media to final assay concentration. 100 uL of each inhibitor were added to cells at the dilution indicated in the plate map below. Cells were incubated with the inhibitors for 1 hour at 37° C.

### EGF Stimulation

Lysis buffer was prepared as according to MSD protocols and stored on ice. Growth factor was also prepared at a 2× concentration in serum-free media. A final concentration of 50 ng/mL EGF was added to each cell well. For starved controls serum-free media was used.

In total, 100 ul of EGF was added to each well at the completion of the inhibitor incubation, except for starved controls, which receive 100 ul serum free media. Samples were incubated at 37° C. for 15 minutes.

### Cell Lysis

Following the 15 minute incubation, media was removed from cells by aspiration. 100 uL of the previously prepared MSD lysis buffer was added to each well of the cell plate. The plate was allowed to shake at room temperature for 10 minutes. While the cells are being lysed, a MSD plate was prepared by washing 4× with 150 uL of Tris Wash Buffer. After washing the MSD plate, 30 uL of the lysed cells were transferred to the washed MSD plate.

### Addition of Antibody

Antibody solution was prepared, kept in the dark and cold, washed MSD plate four times with 150 ul Tris Wash Buffer, tapping onto absorbent towel to remove all traces of wash buffer. 25 ul of antibody solution was added to each well. The MSD plate was washed four times with 150 ul Tris Wash Buffer, tapping onto absorbent towel to remove all traces of wash buffer.

### Read Plate

Prepare MSD Read buffer: 10 ml read buffer into 30 ml DI H₂O Add 150 ul Read buffer to each well, taking care not to generate bubbles Read plate on MSD Sector Imager

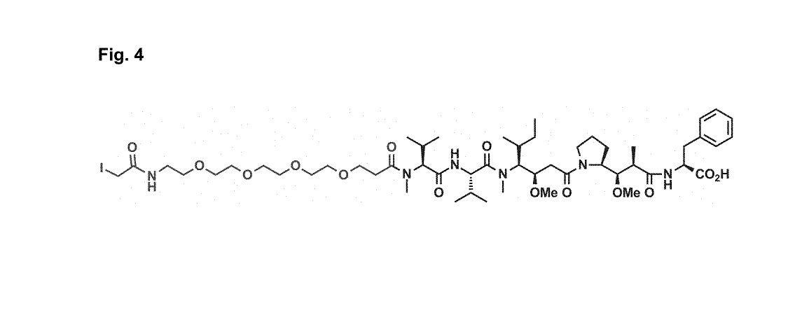

Following the plate read, the data was collected and processed using GraphPad Prism software. Specifically, targeted 83v11-SPION were plotted against its parent FN3 domain molecule (83v2) and non-targeted SPION. As illustrated in the plot graph of FIG. 3, the FN3 domain molecules maintain their activity once they are coupled with the nanoparticles as the 83v11-SPION is able to effectively inhibit EGFR phosphorylation similar to the non-coupled 83v2 FN3 domain molecule.

### Example 6: Intracellular Delivery of Cytotoxic Payload Via EGFR or PSMA-Binding Centyrins

### Reagents and Constructs:

The gene encoding S. aureus sortase A was produced by DNA2.0 and subcloned into pJexpress401 vector (DNA2.0) for expression under the T5 promoter. The sortase construct for soluble expression is lacking the N-terminal domain of the natural protein consisting of 25 amino acids since this domain is membrane associated (Ton-That et al., Proc Natl Acad Sci USA 96: 12424-12429, 1999). The sortase was modified with an N-terminal His6-tag for purification followed by a TEV protease site for tag removal (ENLYFQS, SEQ ID 12). The gene also includes 5 mutations from the natural sequence that have been reported to increase the catalytic efficiency of the enzyme (Chen et al., Proc Natl Acad Sci USA 108: 11399-11404, 2011) (SEQ ID 13). The plasmid was transformed into E. coli BL21 Gold cells (Agilent) for expression. A single colony was picked and grown in Luria Broth (Teknova) supplemented with kanamycin and incubated 18 h at 37° C. 250 RPM. 250 mL of Terrific Broth (Teknova), supplemented with kanamycin, was inoculated from these subcultures and grown at 37° C. for ˜4 h while shaking. Protein expression was induced with 1 mM IPTG, and the protein was expressed for 18 h at 30° C. Cells were harvested by centrifugation at 6000 g and stored at −20 C until purification. The frozen cell pellet was thawed for 30 min at room temperature and suspended in BugBusterHT protein extraction reagent (EMD Millipore) supplemented with 1 uL per 30 mL of recombinant lysozyme (EMD Millipore) at 5 ml per gram of cell paste and incubated for 30 minutes on a shaker at room temperature. The lysate was clarified by centrifugation at 74 600 g for 30 min.

The supernatant was applied onto a gravity column packed with 3 mL of Qiagen Superflow Ni-NTA resin pre-equilibrated with buffer A (50 mM sodium phosphate buffer, pH 7.0 containing 0.5 M NaCl and 10 mM imidazole). After loading, the column was washed with 100 mL of Buffer A. The protein was eluted with Buffer A supplemented with 250 mM imidazole and loaded on a preparative gel-filtration column, TSK Gel G3000SW 21.5×600 mm (Tosoh) equilibrated in PBS (Gibco). The gel-filtration chromatography was performed at room temperature in PBS at flow rate 10 ml/min using an AKTA-AVANT chromatography system. Purified sortase was then digested with TEV protease to remove the His6 tag. 28 mg of sortase was incubated in 10 mL with 3000 units of AcTEV protease (Invitrogen) in the supplied buffer supplemented with 1 mM DTT for 2 hours at 30 C. The tagless sortase was purified with Ni-NTA resin. The reaction was exchanged into TBS buffer (50 mM Tris pH 7.5, 150 mM NaCl) using PD-10 columns (GE Healthcare) and applied onto a gravity column packed with 0.5 mL of Qiagen Superflow Ni-NTA resin pre-equilibrated with buffer A. The flowthrough was collected and the resin was washed with 3 mL of buffer A which was added to the flowthrough. Flowthrough containing sortase was concentrated to ˜0.5 mL in an Amicon 15 concentrator with 10 kDa cutoff (EMD Millipore). Additional TBS buffer was added and the sample was concentrated again (repeated twice) to exchange the buffer to TBS. ⅓rd volume of 40% glycerol was added (final concentration of 10% glycerol), and the sortase was stored at ˜20° C. for short term use or ˜80° C. for long term.

### Large-Scale Expression, Purification and Conjugation of Centyrins

Centyrins that bind to PSMA or EGFR were discovered as described previously or above (US2014/0155326A1) and cloned into the pET15b vector for expression under the T7 promoter or produced by DNA2.0 and subcloned into pJexpress401 vector (DNA2.0) for expression under the T5 promoter. The resulting plasmids were transformed into E. coli BL21 Gold (Agilent) or BL21DE3 Gold (Agilent) for expression. A single colony was picked and grown in Luria Broth (Teknova) supplemented with kanamycin and incubated 18 h at 37° C. 250 RPM. One liter Terrific Broth (Teknova), supplemented with kanamycin, was inoculated from these subcultures and grown at 37° C. for 4 h while shaking. Protein expression was induced with 1 mM IPTG once the optical density at 600 nm reached 1.0. The protein was expressed for 4 h at 37° C. or 18 h at 30° C. Cells were harvested by centrifugation at 6000 g and stored at −20 C until purification. The frozen cell pellet (˜15-25 g) was thawed for 30 min at room temperature and suspended in BugBusterHT protein extraction reagent (EMD Millipore) supplemented with 0.2 mg/ml recombinant lysozyme (Sigma) at 5 ml per gram of cell paste and incubated for 1 h on a shaker at room temperature. The lysate was clarified by centrifugation at 74 600 g for 25 min. The supernatant was applied onto a 5 ml Qiagen Ni-NTA cartridge immersed in ice at a flow rate of 4 ml/min using an AKTA AVANT chromatography system. All other Ni-NTA chromatography steps were performed at flow rate 5 ml/min. The Ni-NTA column was equilibrated in 25.0 ml of 50 mM Tris-HCl buffer, pH 7.0 containing 0.5 M NaCl and 10 mM imidazole (Buffer A). After loading, the column was washed with 100 ml of Buffer A, followed by 100 ml of 50 mM Tris-HCl buffer, pH7.0 containing 10 mM imidazole, 1% CHAPS and 1% n-octyl-β-D-glucopyranoside detergents, and 100 ml Buffer A. The protein was eluted with Buffer A supplemented with 250 mM imidazole and loaded on a preparative gel-filtration column, TSK Gel G3000SW 21.5×600 mm (Tosoh) equilibrated in PBS (Gibco). The gel-filtration chromatography was performed at room temperature in PBS at flow rate 10 ml/min using an AKTA-AVANT chromatography system.

As a measure of receptor mediated intracellular delivery, Centyrin conjugates were prepared by linking a microtubule disrupting drug to PSMA or EGFR binding Centyrins. Since the microtubule disrupting drug is active in the cytoplasm, efficient intracellular delivery can be assessed using a cytotoxicity assay. Anti-PSMA Centyrins were conjugated to vc-MMAF through a sortase tag. For conjugation to the sortase tag, bacterial pellets were thawed, resuspended and lysed in BugBusterHT (EMD Catalog #70922) supplemented with recombinant human lysozyme (EMD, Catalog #71110). Lysis proceeded at room temperature with gentle agitation, after which the plate was transferred to a 42° C. to precipitate host proteins. Debris was pelleted by centrifugation, and supernatants were transferred to a new block plate for sortase-catalyzed labeling. A master mix containing Gly3-vc-MMAF (Concortis), tagless SortaseA, and sortase buffer (Tris, sodium chloride, and calcium chloride) was prepared at a 2× concentration and added in equal volume to the lysate supernatants. The labeling reaction proceeded for two hours at room temperature, after which proteins were purified using a Ni-NTA multi-trap HP plate (GE Catalog #28-4009-89). Protein conjugates were recovered by step elution with imidazole-containing elution buffer (50 mM Tris pH7.5, 500 mM NaCl, 250 mM imidazole), filter sterilized and used directly for cell based cytotoxicity assays.

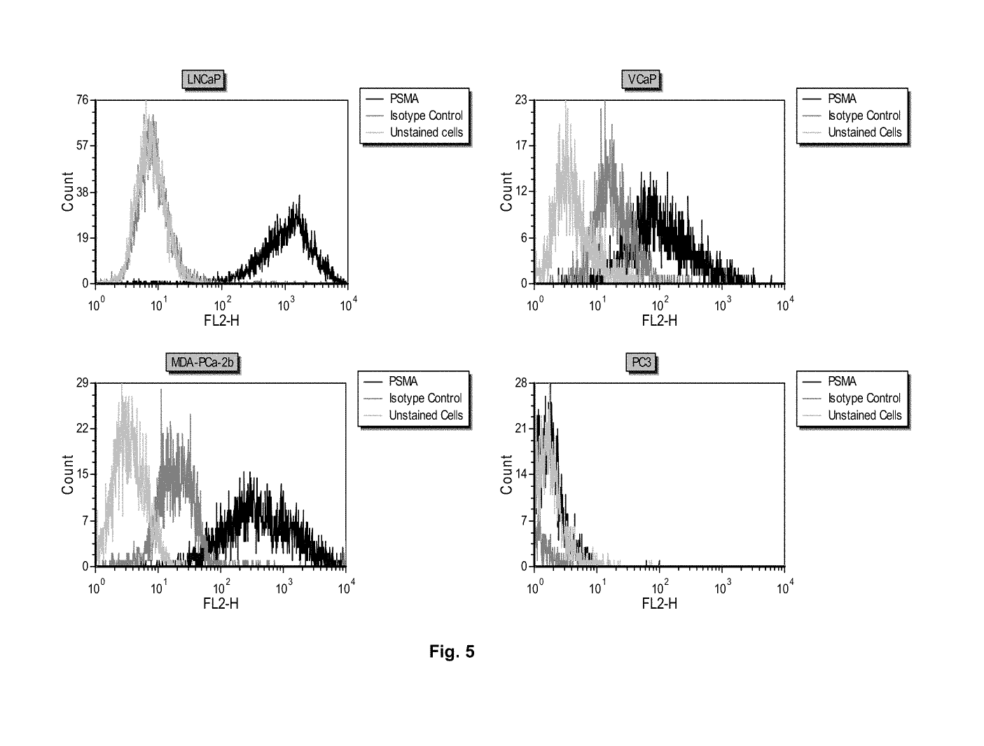

Bivalent anti-EGFR Centyrins were encoded by genetically linking two copies of anti-EGFR Centyrin, P155R8-G3 (SEQID 1) separated with a peptide linker to an albumin binding domain (ABDcon12, SEQID 7) (US2013/0316952A1) with one to four cysteine residues. Centyrins, listed in Table 5, were conjugated to Iodoacetyl-PEG4-MMAF (IAA-MMAF, FIG. 4, Concortis). For conjugation, Centyrins were diluted to 100 uM in PBS and reduced with 10 mM TCEP. Proteins were then precipitated with ammonium sulfate, washed with additional ammonium sulfate to ensure TCEP removal and re-dissolved in PBS. Reduced protein was reacted with a 4- to 8-fold excess of IAA-MMAF per cysteine in 100 mM borate buffer (pH=9.0) for 4.5 h at room temperature. Reaction was quenched by adding N-ethyl maleimide to cap any unreacted cysteines and proteins were purified on Nickel NTA Superflow resin (Qiagen) by gravity flow, exchanged into PBS with Zeba columns (Thermo); endotoxin was removed with EndoBindR resin (BioDTech).

TABLE 5
EGFR Centyrin drug conjugates

| | | | # MMAF after |
|---|---|---|---|
| Centyrin | SEQ ID | Binding antigen | conjugation |
| EEB8 | 2 | EGFR | 1 |
| EEB9 | 3 | EGFR | 2 |
| EEB11 | 4 | EGFR | 3 |
| EEB10 | 5 | EGFR | 4 |
| ECB166 | 6 | None (untargeted) | 3 |

### Quantification of PSMA Expression in Cell Lines

Prostate cancer cell lines (LNCaP, VCAP, MDA-PCa-2B, and PC3) were obtained from ATCC (Manassas, Va.) and cultured using recommended growth media. PSMA expression level was quantified by flow cytometry. Cells were lifted from substrate with Accutase (MP Biomedicals, Santa Ana, Calif.) and stained with saturating levels of anti-PSMA antibody conjugated to PE (Clone GCP-05, 20 ug/mL, Abcam, Cambridge, Mass.) or isotype control (R&D Systems, Minneapolis, Minn.) for 1 h. Excess antibody was rinsed away and fluorescence was recorded using a BD FACs Calibur. Antibody binding was quantified using Quantibrite beads (BD Biosciences, San Jose, Calif.) as directed by the manufacturer. PSMA expression was determined by subtracting background signal from the iostype control from the signal measured for the PSMA specific antibody (assuming each antibody can bind 2 PSMA receptors). FIG. 5 shows representative histograms and Table 4 summarizes PSMA quantification across the four cell lines.

TABLE 6
PSMA levels in prostate cancer cell lines.

| | PSMA expression (mean ± |
|---|---|
| | standard deviation) |
| LNCAP | 187K ± 103K |
| MDA-PCa-2b | 114K ± 2K |
| VCaP | 45K |
| PC3 | antigen negative |

### Selective Cytotoxicity of Anti-PSMA Centyrin Drug Conjugates on PSMA+ Cells

Cytotoxicity of Centyrin-vcMMAF conjugates was assessed in LNCaP, VCAP, MDA-PCa-2B, and PC3 cells in vitro. Cells were plated in 96 well black plates for 24 h and then treated with variable doses of Centyrin-vcMMAF conjugates. Cells were allowed to incubate with Centyrin drug conjugates (CDCs) for 66-72 h. CellTiterGlo (Promega, Madison, Wis.) was used to assess toxicity, according to manufacturer's instructions. Luminescence values were imported into Excel and pasted into Prism for graphical analysis. Data were transformed using X=Log(x), then analyzed using nonlinear regression, applying a 3-parameter model to determine IC50.

Table 7 shows IC50 values for several Centyrins conjugated to vcMMAF across a panel of cell lines. CDCs were most potent in LNCaP cells, a line known to express high levels of PSMA. CDCs were also active in MDA-PCa-2B and VCAP cells, prostate cancer lines with lower levels of PSMA. No activity was observed in PC3 cells, a PSMA negative cell line, demonstrating selectivity. IC50s correlated with the number of antigen present on each cell, with best activity observed on LNCaPs, the cell line with the most PSMA expression.

TABLE 7
Cytotoxicity of PSMA-binding Centyrin drug conjugates. Data represent averages
between one and nine curve fits. Data are presented as average ± SEM.

| | | Cytotoxicity Assays of Centyrin-Drug-Conjugates | | | |
|---|---|---|---|---|---|
| | | LNCaP | MDA-PCA-2B | VCAP | PC3 |
| | SEQ | cells IC₅₀ | cells IC₅₀ | cells IC₅₀ | cells IC₅₀ |
| CENTYRIN | ID | (nM) | (nM) | (nM) | (nM) |
| P233FR9P1001-H3-1 | 8 | 0.4 | 4.6 ± 1.2 | 15.2 ± 1.0 | >500 |
| P234CR9_H01 | 9 | 22.6 | 150.8 ± 4.4 | 401.0 ± 130.0 | >500 |
| P233FR9_H10 | 10 | 0.5 ± 0.1 | 5.8 ± 2.3 | 25.9 ± 15.0 | >500 |
| P229CR5P819_H11 | 11 | 9.3 ± 1.9 | 106.8 ± 13.6 | 231.0 ± 38.0 | >500 |

### Cytotoxicity of Anti-EGFR Centyrin Drug Conjugates on EGFR+ Cells

Cytotoxicity of Centyrin-PEG4-MMAF conjugates was assessed in NCI-H292 and NCI-H1573 cells in vitro. Cells were plated in 96 well black plates for 24 h and then treated with variable doses of Centyrin-PEG4-MMAF conjugates. Cells were allowed to incubate with Centyrin drug conjugates (CDCs) for 66-72 h. CellTiterGlo was used to assess toxicity. Luminescence values were imported into Excel and pasted into Prism for graphical analysis. Data were transformed using X=Log(x), then analyzed using nonlinear regression, applying a 3-parameter model.

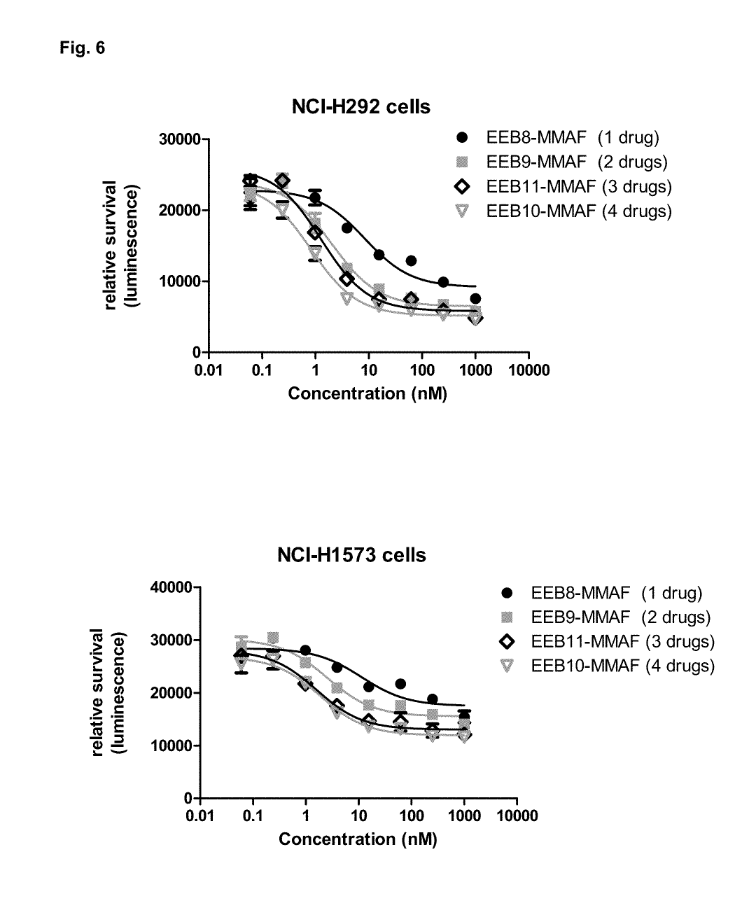

FIG. 6 shows relative cell survival following treatment of Centyrin-drug conjugates with 1, 2, 3, or 4 drug molecules in either A) NCI-H292 or B) NCI-H1573 cells. All conjugates resulted in cell death and the degree of toxicity correlated with the number of drugs per molecule.

### In Vivo Efficacy in 11292 Tumor Xenografts

NCI-H292 cells were grown in RPMI+10% FBS and subcutaneously implanted (2×10⁶ cells diluted in 50% matrigel) into female Charles River SCID beige mice (n=8/group). When tumors reached ˜200 mm³, mice were dosed intravenously with 4.7 mg/kg Centyrin or Centyrin-drug conjugate. Each Centyrin contained an albumin binding domain for half-life extension. A total of three doses were injected into mice over the course of one week on days 1, 3, 6. Tumors were measured twice weekly using calipers sacrificed when tumors were greater than 2000 mm³. Group tumor measurements are reported when more than 4 or more mice remained.

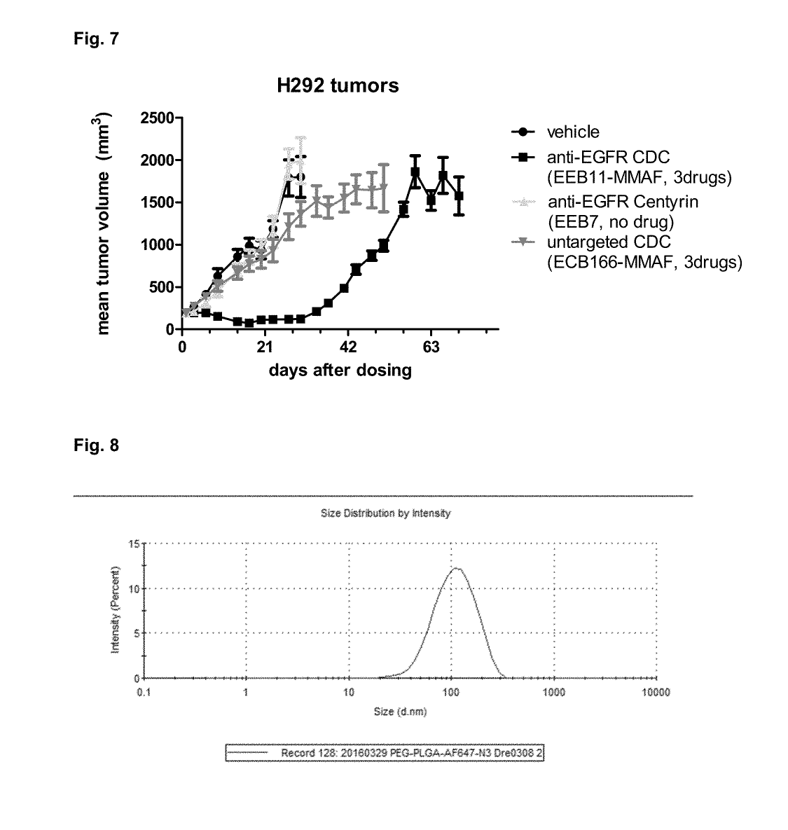

FIG. 7 shows mean tumor volumes for each treatment group. Neither untargeted Centyrin-drug conjugate nor unconjugated Centyrin significantly impacted tumor volume. In contrast, profound tumor growth suppression was seen in mice treated with anti-EGFR Centyrin-drug conjugate. Tumor regression or growth suppression was observed longer than four weeks beyond the initiation of dosing for the group treated with anti-EGFR Centyrin drug conjugate.

### Example 7: Intracellular Delivery of PEG-PLGA Nanoparticles by Targeting with EGFR Binding Centyrin

Poly(lactic-co-glycolic acid) (PLGA) nanoparticles (NPs) display biocompatibility and efficacy, owing to their success in treating infectious diseases and cancer [1-7]. PLGA compared to poly(gamma-glutamic acid) (PGA) or poly-lactic acid (PLA) displays greater hydrolytic stability and faster degradation rate. While there has been some success for siRNA knockdown and therapeutic efficacy by PLGA NPs, there still exists the barrier for specific targeting and intracellular endosome-escape which must be overcome to improve potency. PEG-PLGA NPs have been used for the delivery of small molecule chemotherapeutic drugs as well as nucleic acids [8, 10]. The application of targeting for PEG-PLGA NPs can be used to deliver drug and nucleic acid cargo, making the system versatile in nature [11, 12].

Synthesis of Centyrin-Functionalized PEG-PLGA NPs

The steps to synthesize Centyrin-functionalized PEG-PLGA NPs are as follows:

1) Functionalize a small percentage of amines on PEG-PLGA nanoparticles with a fluorophore-AF647

2) Functionalize the rest of the amines with azides

3) Consume any (unreacted) remaining amines with succinic anhydride

4) Purify PEG-PLGA nanoparticles via desalting column to remove unreacted fluorophore and azide

5) Conjugate Centyrins with Mal-PEG4-DBCO and by click chemistry conjugate Centyrins to the surface of PEG-PLGA-Centyrin nanoparticles

6) Purify final product via Superdex 200 column to remove unreacted Centyrin Below is a detailed outline of the steps above.

1) Functionalize amines with a fluorophore. 30 mol % NH2-PEG-PLGA (Phosphorex, Inc.) will be diluted in 1:1 in 50 mM Sodium Bicarbonate buffer, pH 8.5. Amine reactive fluorophore will be added such that the final molar ratio of amines to fluorophore is 1:1. In 1 ml of PEG-PLGA at 1.4 mg/m, there is 1.4 mg's or 93.3 nanomoles of NPs. The percentage of amines in the sample is 28 nanomoles of amines in the sample (93.3 nmoles*30% aminated). To achieve 28 nanomoles of AF647 from a 20 mM stock, add 1.4 uL to the 1 mL suspension. The reaction was incubated on a thermomixer at room temperature for 30 minutes, shaking at 900 rpm.

2) Functionalize remaining amines with NHS-PEG4-Azide. This reaction from step 1 does not need to be purified before adding the azide. To ensure completion of reaction, the azide is added at a 10-fold molar excess to the original amine content. Calculations for azide addition are:
28 nanomoles*10-fold molar excess=280 nanomoles of azide needed
280 nanomoles/100 mM=2.8 uL of azide sample to be directly added to nanoparticle suspension

Add 2.8 uL of the azide crosslinker to the nanoparticle reaction and allow the reaction to run at room temperature for 1 hour, shaking at 900 rpm.

3) Succinic Anhydride is added to remove any residual amines. Add 1 uL of 1 M succinic anhydride to the PEG-PLGA-AF647-Centyrin reaction and incubate for 30 minutes at room temperature on a thermomixer shaking at 950 rpm.

4) PEG-PLGA-AF647-Centyrin Purification by Desalting. The AKTA Avant which currently has a GE HiPrep 26/10 desalting column is used for this step.

5) Centyrin Functionalization with DBCO-PEG-Maleimide, followed by click reaction with Centyrin-DBCO and NP-azide.

a. Centyrin functionalization steps following routine reduction, precipitation and reaction with the crosslinker. The Centyrin-DBCO conjugate is concentrated on a 10 kDa MWCO Amicon to 7-10 mg/mL final concentration. For calculations for this step, it is assumed that there are 30 mol % azides to ensure that enough of the Centyrin-DBCO is added. Additionally, it is recommended to add ˜3-fold molar excess of the DBCO product to the nanoparticles. There is 28 nanomoles of azide (30 mol % azide from a 1.4 milligram prep). If the Centyrin-DBCO is at 7 mg/mL (˜570 uM), 84 nanomol of Centyrin will be added to achieve 3-fold molar excess (84 nanomol/570 uM=147 uL of Centyrin to be added). The GE HiPrep 26/10 desalting column for purification of excess DBCO crosslinkers from conjugated Centyrins will be used.

b. Centyrin/PEG-PLGA Click reaction. 50-100 kDa MWCO Amicon is used to bring the PEG-PLGA particles back to their original volume (2500x rcf spin for 5-10 minutes is usually enough). Take 1 mL of nanoparticle sample and transfer to a fresh Eppendorf tube. The reaction can be carried out at room temperature for ˜4 hours or at 4-10 C overnight. The former option was chosen.

6) Purification of final product

The small prep grade Superdex 200 column is used to separate the components of mixture of targeted NPs from unreacted Centyrin-DBCO. The fractions containing NP will be collected, pooled and used directly for experiments. A loss of 20% of NP yield is assumed. The fluorescence of all NPs will be compared and normalized to ensure the fluorescence is accurately indicative of concentration.

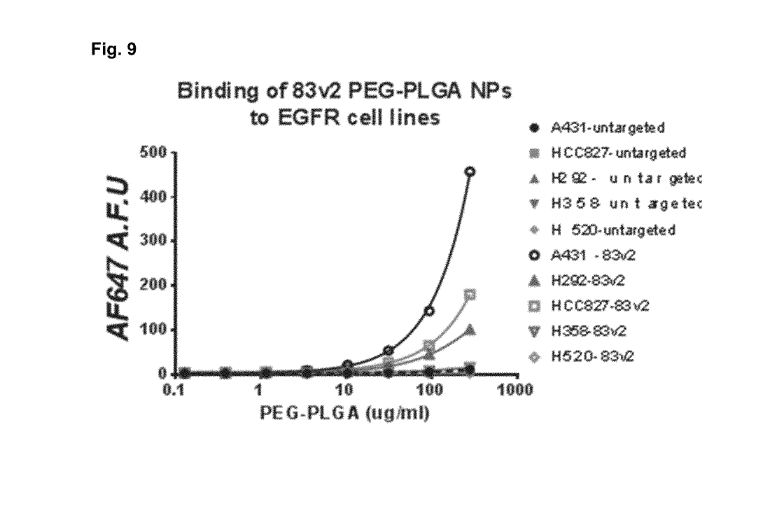

(FIG. 8: Size distribution of AF647-labeled PEG-PLGA- NP post SEC purification)

Quantitative Assessment of Binding and Internalization of Centyrin-Targeted PEG-PLGA NPs

PEG-PLGA Nanoparticles were functionalized with 83-Centyrin. The NP integrity and size is confirmed by dynamic light scattering (FIG. 8). NP size range was 100-120 nm (z-average diameter).

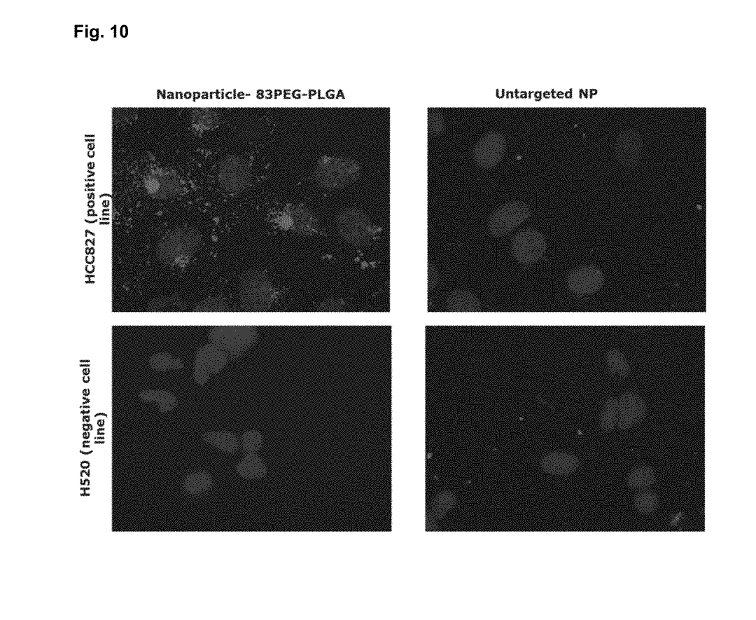

The specificity of targeted NP binding to the receptor was assessed by measuring the relative binding of 83-targeted PEG-PLGA NPs to a panel of EGFR-expressing cell line with varying receptor density (panel includes negative cell line H520). The control NPs include untargeted (No Centryin) NPs. The cell lines obtained from ATCC were Epidermoid Carcinoma A431 (>1,000,000 receptors/cell), H358 bronchioalveolar carcinoma (˜100k receptors/cell), Lung Adenocarcinoma HCC827 (˜800k receptors/cell), H292 Lung Adenocarcinoma (˜200k receptors/cell), and H520 Lung Carcinoma (negative cell line). The procedure involves obtaining cells at 1 e6 cells/ml in 100 ul in RPMI media and adding NPs with incubation at 37° C. for 1 hour. The plate was put under rotation. Following incubation, samples were washed and resuspended in FACS buffer for analysis by Flow Cytometry. Each of the cell line was gated by FSC/SSC. The gate (region of cells considered viable and appropriate for analysis) for each cell line was the same. Cells were analyzed for AF647 by the FL-4 channel. Increased AF647 signal indicates greater binding and internalization events. FIG. 9 shows dose-dependent binding and internalization of the 83-Centyrin targeted NPs to the EGFR-expressing cell lines. Further, this dose-dependent binding is correlated to EGFR receptor density. The targeted NP do not show binding to the negative, non-EGFR expressing cell line, H520. Further, the untargeted NP do not display any binding to the cell lines.

FIG. 9 shows receptor density dependent and dose-dependent binding and internalization of AF647 labeled 83-Centyrin targeted PEG-PLGA NPs after 1 hour, 37° C. incubation on a panel of EGFR-expressing cell lines. FACS was used to quantitatively measure AF647 signal from Centyrin-NPs

Qualitative Assessment of Binding and Internalization of Centyrin-Targeted PEG-PLGA NPs by Imaging

The purpose of this experiment is to examine internalization of Centyrin-labeled NPs via imaging EGFR+ cell lines using the Opera confocal imaging system. Specifically, fluorescent 83v2-PEG-PLGA NPs will be incubated with A431, HCC827, H292, H358 and H520 (the same panel of cells used for FACS-based quantification). Each of the five cell lines was cultured in a T150 flask and was pulled from a 37° C. incubator. Media for cells was RPMI+10% FBS. Cell media was removed and the flasks were gently rinsed with 1× dPBS. Following this, cells were dissociated using a quick exposure to 5 mL 0.25% trypsin until the cells were found to have dislodged from their T150 flask bottom (approximately 2-7 minutes). Upon observing loose cells, 8 mL of cell media (RPMI 1640+Glutamax, 10% FBS) was added to the flask. The cells were transferred to a 15 mL conical tube and spun at 1000 rpm for 5 minutes at RT. The supernatant in the tube was aspirated and the remaining cells were resuspended in 5 mL of RPMI 1640 buffer for counting. A cell count was performed and the cells were diluted to various concentrations in the 96-well plate. Cell density of 10000 cells/well was chosen such that cells will cover 70% of the surface after 20 hours of incubation prior to treatment. The next day media was aspirated, diluted samples were added to their wells first and volumes were brought up to 100 μL using RPMI 1640 cell media. Samples were incubated at 37° C. for 4 hours covered to prevent photobleaching. Following the incubation time, plates were spun at 4′C for 3 minutes at 1500 rpm, washed 2× with cold FACS buffer. Cells with Hoechst stain for ˜20 mins to allow nuclear staining. Following this, cells were washed 1× and resuspended in 150 ul of FACS buffer and then imaged.

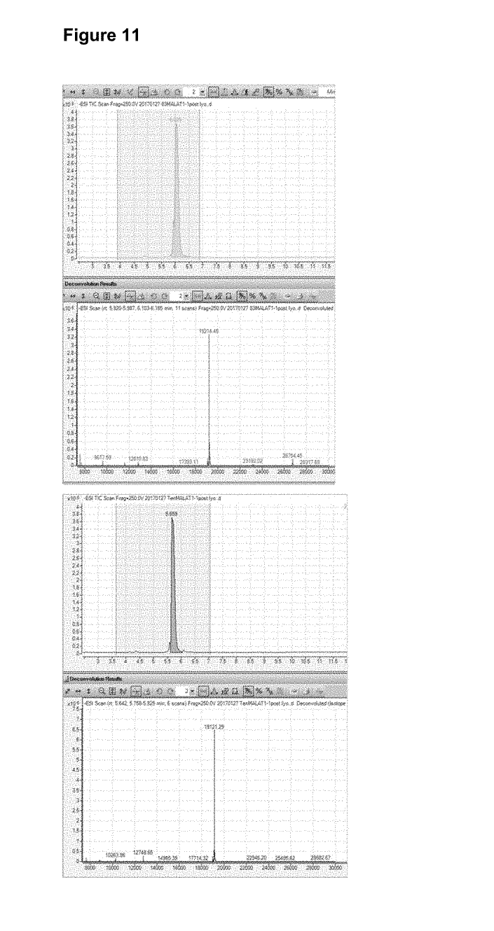

FIG. 10: Cellular binding and internalization of AF647 labeled-83-Centyrin (60×) shows Centyrin specific binding to EGFR-expressing cell line, HCC827. 20×: C2f3, C5f1, C8f2; 60×: C2f7, C5f2, C8f

Blue=100-2000(0.5), Red=10-100(0.5), Red reduced=10-2000(0.5)

The imaging data confirms binding and internalization of 83 Centyrin-targeted NP to the EGFR-expressing cell line, HCC827. The targeted NPs do not show binding to the negative cell line, H520, confirming specificity. Further, the untargeted NPs do not show binding to the HCC827 cell line, or the negative cell line. The lower-receptor number cell line, H292, shows much less AF647 signal.

### Example 8: Preparation and Selective Interacellular Delivery of EGFR Centyrin Conjugated Antisense Oligonucleotides (ASO)

Centyrins can be used to target oligonucleotides specifically to the target cell of interest by the receptor-mediated uptake. As described herein, several Centyrins have been conjugated to 2nd generation 2′Omethyl gapmer ASO and the resulting conjugates characterized for in vitro gene knockdown. Centyrins include targets to EGFR receptor and CD8 receptor. The goal of targeted uptake of ASOs with Centyrins will be to reduce the non-specific PS-ASO mediated uptake and enhance potency of ASO by receptor-mediated uptake.

Methods:

Synthesis and Characterization of Centyrin-ASO Conjugates

TABLE 8
MALAT1 targeted ASOs with click handle
and gapmer configuration

ASO Sequence Source
MALAT1-seq. 1
/5DBCON/mU*mG*mC*mC*mU*T*T*A*G*G*A*T*T*C*T*mA*mG*
mA*mC*mA (Integrated DNA Technologies (IDT))
(SEQ ID NO: 47)
MALAT1-seq. 2
mC*mC*mA*mG*mG*C*T*G*G*T*T*A*T*G*A*mC*mU*mC*mA*mG
(Integrated DNA Technologies (IDT)) (SEQ ID NO: 48)
m = 2′Omethyl,
*indicates phosphorothioate linkage. DBCO and ASO are
linked by Phosphodiester linkage (absence of *)

Conjugates were synthesized by click chemistry conjugation with DBCO end group at the 5′ end of the MALAT1 ASO and Centyrin with azide functional group at C-term. The 2′O methyl PS ASO in gapmer configuration was obtained from Integrated DNA Technologies (IDT) and Centyrin was expressed and purified according to Goldberg et al. [1]. The amounts of ASO added to each of the protein were calculated based on a single ASO to two Centyrin molar ratio with a starting ASO mass of ˜2.0 mg. For the Tencon-MALAT1 ASO, anion exchange CaptoQ column was used for purification. 1 mL CaptoQ column was prepared on the AKTA Avant (HiTrap CaptoQ lml, GE healthcare life sciences, 17-5470-51). Details are column volume of 0.962 ml, buffer A at 20 mM Tris pH 6.5, and buffer B at 20 mM Tris pH 6.5, 2M NaCl. The method used for purification: a. Equilibrate column with 5CV of Buffer A at 1 ml/min, b. Inject sample using sample pump, c. elute samples using a step gradient of 20% B (5CV), linear gradient to 70% B over 35CV, step gradient at 100% (5CV) and finally a step gradient at 0% (8CV), d. Collect all fractions 1 mL in a 96-well plate. The fractions were analyzed by negative ion LC-MS to analyze to determine ASO alone, ASO conjugate and Centyrin fractions. The relevant fractions with conjugate material were pooled and buffer exchanged by dialysis for 4 hours against 1 mM sodium phosphate (pH 7.0), frozen overnight at −80 C and lyophilized for 2-3 days. For the 83-MALAT1 ASO, a two-step method of purification was employed with anion exchange CaptoQ column first, followed by his-trap method of purification with elution at high imidazole concentration. The anion method of purification is the same as for Tencon-MALAT1 conjugate. The his-trap method of purification utilized a HiTrap blue HP lml (GE healthcare life sciences, 17-0412-01), with column volume of 0.962 ml, buffer A at 50 mM Tris pH7.5, 500 mM NaCl, 10 mM imidazole, and buffer B at 250 mM imidazole. The elution protocol includes 100% buffer B over 20 CV, followed by a step gradient of 100% B over 5CV with 1.0 ml fractions collected in a 96-well deep plate. The analysis of fractions collected, dialysis, and lyophilization methods are detailed above. For the CD8-368-MALAT1 ASO, a one-step purification by his-trap was employed with details listed above. For this conjugate, the fractions are pooled, dialyzed and lyophilized as described above.

The crude product, fractions from purification, and final purified material was characterized by LC-MS negative ion for accuracy of molecular weight. Analysis was performed on Agilent Model G6230 MS-TOF mass spectrometer. The instrument was operated in negative electro-spray ionization mode and scanned from m/z 700 to 3200. LC conditions were performed on Agilent Infinity II 1290 instrument and included: Waters) (Bridge C8, 2.5 μm particle, 2.1×50 mm; column temperature of 75° C., Buffer A=25 mM Triethylamine, 570 mM 1,1,1,3,3,3-hexafluoro-2-propanol, 10% methanol, Buffer B=acetonitrile; flow rate=0.3 mL/min; gradient 0-2 min 1% B, 2-12 min 1 50% B, 12-15 min 75% B. Mass Spectrometer Instrument settings included: negative polarity, 700-3200 amu mass range; spray voltage 3750 V; source temperature 350° C.; drying gas flow 12 l/min; nebulizer 35 psi; sheath gas 300° C.; sheath gas flow 11 l/min; nozzle voltage 1200V; fragmentor 250V. The MS spectrum was manually de-convoluted using max entropy algorithm between 7000-33000 Da. All protein conjugates were analyzed on negative ion LC-MS. The LC-MS spectra showed for Tencon-MALAT1 ASO at 5.659 min with mass of 19121.29 Da (6.5e6), 83-MALAT1 ASO at 6.02 min with mass of 19214.45 Da (3.2e6), and for CD8-368 MALAT1 ASO at 4.851 min with mass of 7574.05 Da (2e4) and 6.094 min with mass of 18318.5 Da (1e7).

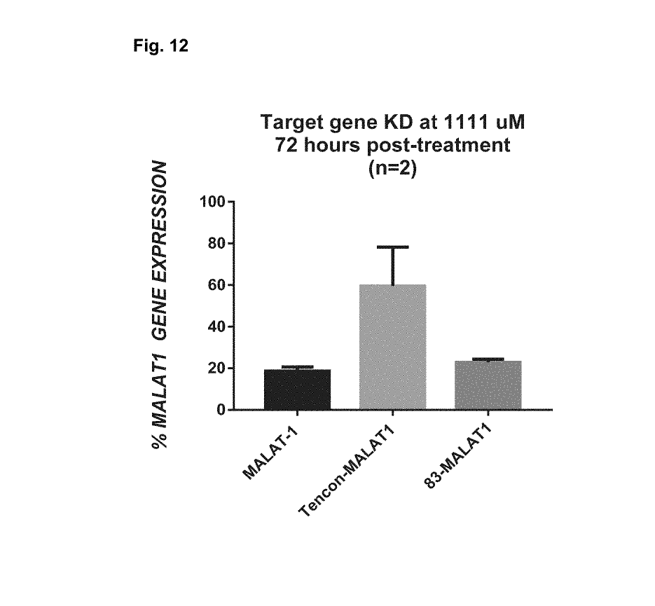

FIG. 11: A) LC-MS of MALAT1-83 conjugate showing accurate molecular weight and single species at 19214 Da, B) LC-MS of MALAT1-Tencon conjugate at 19121.3 Da.

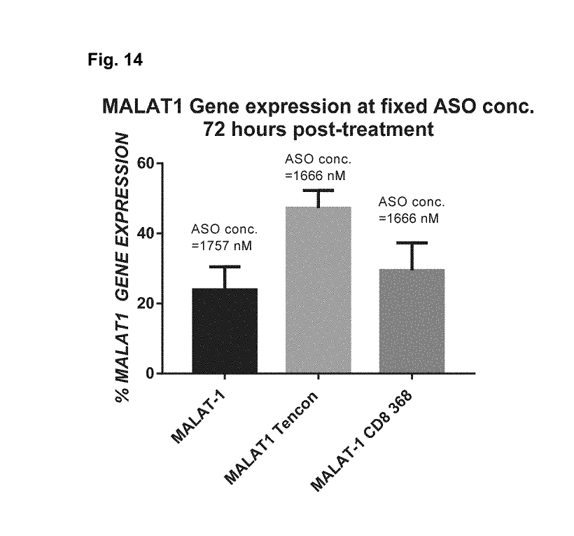

FIG. 13: LC-MS of MALAT1-CD8 368 conjugate at 18318.5 Da (theoretical mass=18320 Da).

MALAT1 mRNA Silencing in A431 Cells Using Centyrin-MALAT1 ASO conjugates

A431 were obtained from Janssen's cell banking system and grown in RPMI with Hepes and 10% FBS. Cells were seeded in 96-well flat bottom plates at 2500 cells/well for 24 hours before treatment with ASO and Centyrin-ASO conjugates. Cells of passage 6 and 7 were used with viability greater than 90%. For PCR, cells were lysed using PLA kit (Protein Quant Sample Lysis Kit (PLA), 25 mL. #Part no. 4448536, lot. no. 00358254, expiry: 2017-02-02). For cDNA synthesis, 2 ul of lysate was mixed with 18 ul of cDNA synthesis mix (High Capacity cDNA Reverse Transcription Kit, Life Technologies: 10×RT (Part no. 4368813 lot no. 002837). Once cDNA was obtained, it was stored at 4 C and used for PCR step. For PCR, samples were analyzed in duplicate in a 384-well plate using viiA7. For PCR, 2.5 ul of cDNA was mixed with 7.5 ul of PCR mix (TaqMan® fast Gene Expression Master Mix #4444557, Life Technologies, lot1504065, expiry 24 Apr. 2016) and primers for PPIB (20×PPIB Vic primer-Applied Biosystems #Hs00168719 PN 4448490 750 ul 20×, lot 150715-001 H03, Exp. July 2017), housekeeping gene, and MALAT1 (20×MALAT1 FAM labeled primer, PN 4351370, exp. January 2022 68G12, lot. P170113-003 G11), target gene were amplified. The qPCR Cycling Parameters were 40 cycles in the order of 1 cycle at 50° C. for 120 seconds, 94.5° C. for 10 minutes, 95° C. for 15 seconds, followed by 60° C. for 1 minute. The delta delta Ct method was used to measure gene knockdown of MALAT1, with 30% CV criteria applied for technical replicates. The threshold was set to 0.2 (exponential phase of amplification curve).

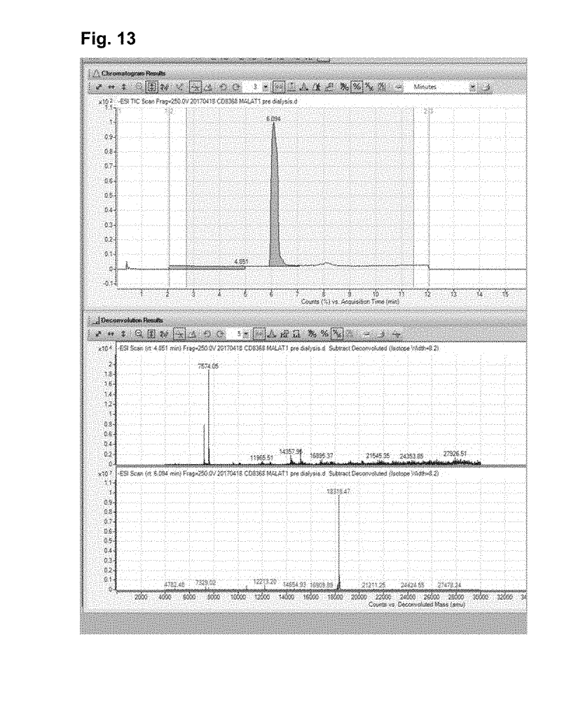

FIG. 12: MALAT1 gene expression measured by rt-PCR in A431 cells treated with ASO or Centryin-ASO by free uptake. PCR was measured 72 hours post-treatment. Replicates from two biological experiments were averaged.

For MALAT1 KD with CD8-368 targeted Centyin, activated primary T cells were used. Tcells were obtained from Janssen's cell banking system and grown in RPMI with Hepes and 10% heat-inactivated FBS. Human CD3+ T cells (obtained from hema Care, cat PBO8NC-3, lot 17041560) were activated every 1-2 weeks using IL-2 (final concentration of 20 U/ml). Cells were of 90% viability were seeded and treated on the same day (since suspension cells). The cell seeding density of 100,000 cells/well was used to ensure sufficient PCR signal (mRNA content in T cells is lower compared to other cell lines). The method of PCR, were similar to that of A431 cells with the exception that housekeeping gene was gapdh (20×gapdh 20×GapDH VIC Labeled Primer Hs02758991_g1, Applied Biosystems).

FIG. 14: MALAT1 gene expression measured by rt-PCR in primary T cells treated with ASO or Centryin-ASO by free uptake. PCR was measured 96 hours post-treatment. Data was captured from a single experiment.

### Example 9: Selection and Characterization of Centyrins that Bind to Human EpCam and their Conjugates

Panning for Centyrins that Bind to Human EpCAM

CIS display was used to select EpCAM-binding Centyrins from the TCL7, TCL9, and TCL14 libraries. For in vitro transcription and translation (ITT), 3 μg of library DNA were incubated at 30° C. with 0.1 mM complete amino acids, 1× S30 premix components, and 15 μL of S30 extract (Promega) in a total volume of 50 μL. After 1 hour, 375 μL of blocking solution (1×TBS pH 7.4, 0.01% I-block (Life Technologies, #T2015), 100 ug/ml herring sperm DNA) was added and reactions were incubated on ice for 15 minutes. ITT reactions were incubated with biotinylated recombinant human EpCAM fused to an Fc domain (R&D Systems catalog # XXX) The biotinylated recombinant protein and the bound library members were captured on neutravidin or streptavidin coated magnetic beads. Unbound library members were removed by successive washes with TBST and TBS. After washing, DNA was eluted from the target protein by heating to 85° C. for 10 minutes and amplified by PCR for further rounds of panning. High affinity binders were isolated by successively lowering the concentration of target EpCAM during each round from 400 nM to 100 nM and increasing the washing stringency.

Following panning, selected FN3 domains were amplified by PCR, subcloned into a pET vector modified to include a ligase independent cloning site, and transformed into BL21-GOLD (DE3) (Stratagene) cells for soluble expression in E. coli using standard molecular biology techniques. A gene sequence encoding a C-terminal poly-histidine tag was added to each FN3 domain to enable purification and detection. Cultures were grown to an optical density of 0.6-0.8 in TB medium supplemented with 100 μg/mL carbenicillin in 1-mL 96-well blocks at 37° C. before the addition of IPTG to 1 mM, at which point the temperature was reduced to 30° C. Cells were harvested approximately 16 hours later by centrifugation and frozen at −20° C. Cell lysis was achieved by incubating each pellet in 0.6 mL of BugBuster® HT lysis buffer (Novagen EMD Biosciences) with shaking at room temperature for 45 minutes.

### Cell-Based Screening

The Centyrins identified in panning were also screened for binding to A431 cells, a carcinoma cell line that expresses EpCAM at high levels. Centyrin lysates were diluted 1:100 in 2% FBS-PBS and added to 96-well plates containing 8×104 dissociated A431 cells. After 1 hr incubation on ice, media was removed and cells were resuspended in 100 uL 2% FBS-PBS. The anti Centyrin rabbit antibody pAb25 was then added to 10 ug/mL and incubated on ice for 1 hr. Media was removed and cells were resuspended in 100 uL 2% FBS-PBS, and Donkey-anti-Rabbit-F(ab′)2-PE conjugate (Jackson Immunoresearch catalog #711-116-152) was added at a 1:200 dilution. After 1 hr incubation on ice, media was removed and cells were resuspended in 250 uL 2% FBS-PBS. Binding was analyzed by flow cytometry on a BD FACSCalibur instrument. Centyrins with MFI over 10× over background (cells with pAb25 and secondary antibody only) were classified as cell binders.

Three of the Centyrins from panning against EpCAM were chosen for assessment in targeted delivery, based on the combination of screening data, protein expression, and biophysical characterization (Table X).

Genes encoding the 3 Centyrins, modified with a C-terminal cysteine, were obtained from DNA2.0 and used to express and purify proteins as described above. Centyrins were conjugated to vc-MMAF through cysteine-maleimide chemistry (Brinkley, Bioconjugate Chemistry 3: 2-13, 1992) Cytotoxicity of Centyrin-vcMMAF conjugates was assessed in A431, HT29, and LNCaP cells in vitro. Cells were plated in 96-well black plates for 24 h and then treated with variable doses of Centyrin-vcMMAF conjugates. Cells were incubated with Centyrin drug conjugates (CDCs) for 66-72 h. CellTiterGlo was used to assess toxicity, as described above. Luminescence values were imported into Excel, from which they were copied and pasted into Prism for graphical analysis. Data were transformed using X=Log(x), then analyzed using nonlinear regression, applying a 3-parameter model to determine IC50.

TABLE 9
EpCAM-binding Centyrins tested as drug conjugates

| | | | | Cytotoxicity | |
|---|---|---|---|---|---|
| | | | | Assays of CDCs | HT-29 |
| | SEQ | BC | FG | A431 cells | cells |
| CENTYRIN | ID | (SEQ ID NO) | (SEQ ID NO) | IC50 (nm) | IC50 (nm) |
| ISOP130R5CP6_A06 | 14 | KHYRPGAR | VTALPSYYSSN | 9.9 | 6.7 |
| | | (52) | (54) | | |
| ISOP130R5CP7_G04 | 15 | KHYRPGAR | AAHAIPRYASN | 48.1 | 37.7 |
| | | (52) | (55) | | |
| Iso124R5AB_D6 | 16 | HNHRPQ | AIAVPWNYQSN | 59.2 | 38.3 |
| | | (53) | (56) | | |

An additional panel of 45 Centyrins were later assessed for activity as CDCs. Genes encoding the Centyrins with C-terminal sortase tags were obtained, and proteins were expressed and purified. Centyrins were conjugated to vcMMAF by high-throughput conjugation using the sortase enzyme and Gly3-vcMMAF. Cytotoxicity of the CDCs was assessed in COL0205 cells using the CellTiterGlo assay described above. Each CDC was tested at 3 concentrations (20, 2, and 0.2 nM). 6 CDCs were identified with activity comparable to or more potent than ISOP130R5CP6_A06. These were further characterized in a full dose-response (Table Y). The Centyrins ISOP130R5CP6 F01 and ISOP130R5CP7_G02 were determined to be more potent than ISOP130R5CP6_A06. These, along with ISOP130R5CP6_E08, were also characterized by differential scanning calorimetry to determine melting temperatures.

TABLE 10
Summary of EpCAM binders identified in CDC screen

| | SEQ ID | % viable | | | | Scaffold |
|---|---|---|---|---|---|---|
| COLO205 cells, 1 nM | SEQ ID | Tm (C) | | BC (SEQ ID NO) | FG (SEQ ID NO) | mutation |
| ISOP130R5CP7_G02 | 17 | 39 | 65 | KHYRPGAR | VTHALPTAYTSN | V46I |
| | | | | (52) | (58) | |
| ISOP130R5CP6_E08 | 18 | 66 | 67 | KHYRPGAR | VAALPNNYASN | |
| | | | | (52) | (59) | |
| ISOP130R5CP6_F02 | 21 | 90 | | DQYRKYAG | VTHALPQTYQSN | |
| | | | | (57) | (60) | |
| ISOP130R5CP7_F11 | 22 | 81 | | KHYRPGAR | IWGALPNSYSSN | |
| | | | | (52) | (61) | |
| ISOP130R5CP6_F01 | 20 | 43 | 68 | KHYRPGAR | VTALPSNYISN | |
| | | | | (52) | (62) | |
| ISOP130R5CP6_A02 | 23 | 66 | | KHYRPGAR | VNNALPRWYISN | |
| | | | | (52) | (63) | |
| ISOP130R5CP6_A06 | 19 | 67 | 63 | KHYRPGAR | VTALPSYYSSN | |
| | | | | (52) | (54) | |

### Engineering of Anti-EpCAM Centyrins

Genes encoding “alanine scan” variants of the representative EpCAM-binding Centyrin ISOP130R5CP6_A06 were obtained from DNA2.0 and used to express and purify proteins as described above. Single alanine variants were produced at each of the non-alanine positions in the BC and FG loops, 15 in total. Centyrins were conjugated to vcMMAF via the sortase reaction and purified as described above. COL0205 cells were treated with the conjugates for ˜72 hrs, and cytotoxicity was monitored with CellTiterGlo.

TABLE 11
Alanine-scan variants of ISOP130R5CP6_A06

| Variant | SEQID | IC50 (nM) |
|---|---|---|
| ISOP130R5CP6_A06_K23A | 24 | 1.0 |
| ISOP130R5CP6_A06_H24A | 25 | 2.6 |
| ISOP130R5CP6_A06_Y25A | 26 | 126.9 |
| ISOP130R5CP6_A06_R26A | 27 | 506.6 |
| ISOP130R5CP6_A06_P27A | 28 | 42.6 |
| ISOP130R5CP6_A06_G28A | 29 | 0.8 |
| ISOP130R5CP6_A06_R30A | 30 | 0.8 |
| ISOP130R5CP6_A06_V78A | 31 | 4.5 |
| ISOP130R5CP6_A06_T79A | 32 | 0.5 |
| ISOP130R5CP6_A06_L81A | 33 | 76.0 |
| ISOP130R5CP6_A06_P82A | 34 | 325.5 |
| ISOP130R5CP6_A06_S83A | 35 | 1.4 |
| ISOP130R5CP6_A06_Y84A | 36 | 0.4 |
| ISOP130R5CP6_A06_Y85A | 37 | 564.5 |
| ISOP130R5CP6_A06_S86A | 38 | 1.3 |
| ISOP130R5CP6_A06 | 19 | 1.6 |

Many of the positions tested were tolerant of mutation to alanine, showing little effect on activity. The largest effects were seen at positions Tyr25, Arg26, Pro27, Leu81, Pro82, and Tyr85 suggesting that these amino acids play an important role in EpcAM binding. These residues are largely conserved in the EpCAM binders identified, further supporting their role in binding to EpCAM.

Sequence IDs

SEQ ID No. 1 = P155R8-G3
MLPAPKNLVVSRVTEDSARLSWTAPDAAFDSFQIYYSELLSYGEAIVLTVPGSERSYDLTGLKPGTEYTVSINGVK
GGTRSWSLSAIFTT
SEQ ID No. 2 = EEB8
MLPAPKNLVVSRVTEDSARLSWTAPDAAFDSFQIYYSELLSYGEAIVLTVPGSERSYDLTGLCPGTEYTVSINGVK
GGTRSWSLSAIFTTAPAPAPAPAPLPAPKNLVVSRVTEDSARLSWTAPDAAFDSFQIYYSELLSYGEAIVLTVPGS
ERSYDLTGLKPGTEYTVSINGVKGGTRSWSLSAIFTTAPAPAPAPAPTIDEWLLKEAKEKAIEELKKAGITSDYYF
DLINKAKTVEGVNALKDEILKA
SEQ ID No. 3 = EEB9
MLPAPKNLVVSRVTEDSARLSWTAPDAAFDSFQIYYSELLSYGEAIVLTVPGSERSYDLTGLCPGTEYTVSINGVK
GGTRSWSLSAIFTTAPAPAPAPAPLPAPKNLVVSRVTEDSARLSWTAPDAAFDSFQIYYSELLSYGEAIVLTVPGS
ERSYDLTGLCPGTEYTVSINGVKGGTRSWSLSAIFTTAPAPAPAPAPTIDEWLLKEAKEKAIEELKKAGITSDYYF
DLINKAKTVEGVNALKDEILKA
SEQ ID No. 4 = EEB11
MLPAPKNLVVSRVTEDSARLSWTAPDAAFDSFQIYYSELLSYGEAIVLTVPGSERSYDLTGLCPGTEYTVSINGVK
GGTRSWSLSAIFTTAPCPAPAPAPLPAPKNLVVSRVTEDSARLSWTAPDAAFDSFQIYYSELLSYGEAIVLTVPGS
ERSYDLTGLCPGTEYTVSINGVKGGTRSWSLSAIFTTAPAPAPAPAPTIDEWLLKEAKEKAIEELKKAGITSDYYF
DLINKAKTVEGVNALKDEILKA
SEQ ID No. 5 = EEB10
MLPAPKNLVVSRVTEDSARLSWTAPDAAFDSFQIYYSELLSYGEAIVLTVPGSERSYDLTGLCPGTEYTVSINGVK
GGTRSWSLSAIFTTAPCPAPAPAPLPAPKNLVVSRVTEDSARLSWTAPDAAFDSFQIYYSELLSYGEAIVLTVPGS
ERSYDLTGLCPGTEYTVSINGVKGGTRSWSLSAIFTTAPCPAPAPAPTIDEWLLKEAKEKAIEELKKAGITSDYYF
DLINKAKTVEGVNALKDEILKA
SEQ ID No. 6 = ECB166
MLPAPKNLVVSEVTEDSARLSWTAPDAAFDSFLIQYQESEKVGEAIVLTVPGSERSYDLTGLCPGTEYTVSIYGVK
GGHRSNPLSAIFTTAPCPAPAPAPLPAPKNLVVSEVTEDSARLSWTAPDAAFDSFLIQYQESEKVGEAIVLTVPGS
ERSYDLTGLCPGTEYTVSIYGVKGGHRSNPLSAIFTTAPAPAPAPAPTIDEWLLKEAKEKAIEELKKAGITSDYYF
DLINKAKTVEGVNALKDEILKA
SEQ ID No. 7 = ABDcon12
TIDEWLLKEAKEKAIEELKKAGITSDYYFDLINKAKTVEGVNALKDEILKA
SEQ ID No. 8 = P233FR9P1001_H3-1
LPAPKNLVVSRVTEDSARLSWTAPDAAFDSFPIGYWEWDDDGEAIVLTVPGSERSYDLTGLKPGTEYHVYIAGVKG
GQWSFPLSAIFTT
SEQ ID No. 9 = P234CR9_H01
LPAPKNLVVSRVTEDSARLSWEWWVIPGDFDSFLIQYQESEKVGEAIVLTVPGSERSYDLTGLKPGTEYTVSIYGV
VNSGQWNDTSNPLSAIFTT
SEQ ID No. 10 = P233FR9_H10
LPAPKNLVVSRVTEDSARLSWTAPDAAFDSFAIGYWEWDDDGEAIVLTVPGSERSYDLTGLKPGTEYPVYIAGVKG
GQWSFPLSAIFTT
SEQ ID No. 11 = P229CR5P819_H11
LPAPKNLVVSRVTEDSARLSWDIDEQRDWFDSFLIQYQESEKVGEAIVLTVPGSERSYDLTGLKPGTEYTVSIYGV
YHVYRSSNPLSAIFTT
SEQ ID No. 12 = Sortase A
MSHEIHHHESSGENLYFQSKPHIDNYLHDKDKDEKIEQYDKNVKEQASKDKKQQAKPQIPKDKSKVAGYIEIPDAD
IKEPVYPGPATREQLNRGVSFAEENESLDDQNISIAGHTFIDRPNYQFTNLKAAKKGSMVYFKVGNETRKYKMTSI
RNVKPTAVEVLDEQKGKDKQLTLITCDDYNEETGVWETRKIFVATEVK
SEQ ID No. 13 = tagless Sortase A
SKPHIDNYLHDKDKDEKIEQYDKNVKEQASKDKKQQAKPQIPKDKSKVAGYIEIPDADIKEPVYPGPATREQLNRG
VSFAEENESLDD QNISIAGHTFIDRPNYQFTNLKAAKKGSMVYFKVGNETRKYKMTSIRNVKPTAVEVLDEQKGK
DKQLTLITCDDYNEETGVWETRKIFVATEVK
SEQ ID no. 14 = ISOP130R5CP6_A06_cys
MLPAPKNLVVSRVTEDSARLSWKHYRPGARFDSFLIQYQESEKVGEAIVLTVPGSERSYDLTGLKPGTEYTVSIYG
VVTALPSYYSSNPLSAIFTTGGHEIHHHEGGC
SEQ ID no. 15 = ISOP130R5CP7_G04_cys
MLPAPKNLVVSRVTEDSARLSWKHYRPGARFDSFLIQYQESEKVGEAIVLTVPGSERSYDLTGLKPGTEYTVSIYG
VAAHAIPRYASNPLSAIFTTGGHEIHHHEGGC
SEQ ID no. 16 = IS0124R5AB_D6_cys
MLPAPKNLVVSRVTEDSARLSWHNHRPQFDSFLIQYQESEKVGEAIVLTVPGSERSYDLTGLKPGTEYTVSIYGVA
IAVPWNYQSNPLSAIFTTGGHREIHHHGGC
SEQ ID no. 17 = ISOP130R5CP7_G02srt
MLPAPKNLVVSRVTEDSARLSWKHYRPGARFDSFLIQYQESEKVGEMILTVPGSERSYDLTGLKPGTEYTVSIYGV
VTHALPTAYTSNPLSAIFTTGGHREIHHHGGLPETGGH
SEQ ID no. 18 = ISOP130R5CP6_E08srt
MLPAPKNLVVSRVTEDSARLSWKHYRPGARFDSFLIQYQESEKVGEAIVLTVPGSERSYDLTGLKPGTEYTVSIYG
VVAALPNNYASNPLSAIFTTGGHREIHHHGGLPETGGH
SEQ ID no. 19 = ISOP130R5CP6_A06srt
MLPAPKNLVVSRVTEDSARLSWKHYRPGARFDSFLIQYQESEKVGEAIVLTVPGSERSYDLTGLKPGTEYTVSIYG
VVTALPSYYSSNPLSAIFTTGGHREIHHHGGLPETGGH
SEQ ID no. 20 = ISOP130R5CP6_F01srt
MLPAPKNLVVSRVTEDSARLSWKHYRPGARFDSFLIQYQESEKVGEAIVLTVPGSERSYDLTGLKPGTEYTVSIYG
VVTALPSNYISNPLSAIFTTGGHREIHHHGGLPETGGH
SEQ ID no. 21 = ISOP130R5CP6_F02srt
MLPAPKNLVVSRVTEDSARLSWDQYRKYAGFDSFLIQYQESEKVGEAIVLTVPGSERSYDLTGLKPGTEYTVSIYG
VVTHALPQTYQSNPLSAIFTTGGHREIHHHGGLPETGGH
SEQ ID no. 22 = ISOP130R5CP7_F11srt
MLPAPKNLVVSRVTEDSARLSWKHYRPGARFDSFLIQYQESEKVGEAIVLTVPGSERSYDLTGLKPGTEYTVSIYG
VIWGALPNSYSSNPLSAIFTTGGHREIHHHGGLPETGGH
SEQ ID no. 23 = ISOP130R5CP6_A02srt
MLPAPKNLVVSRVTEDSARLSWKHYRPGARFDSFLIQYQESEKVGEAIVLTVPGSERSYDLTGLKPGTEYTVSIYG
VVNNALPRWYISNPLSAIFTTGGHREIHHIIGGLPETGGH
SEQ ID no. 24 = ISOP130R5CP6_A06_K23A
MLPAPKNLVVSRVTEDSARLSWAHYRPGARFDSFLIQYQESEKVGEAIVLTVPGSERSYDLTGLKPGTEYTVSIYG
VVTALPSYYSSNPLSAIFTTGGHREIHHIIGGLPETGGH
SEQ ID no. 25 = ISOP130R5CP6_A06_H24A
MLPAPKNLVVSRVTEDSARLSWKAYRPGARFDSFLIQYQESEKVGEAIVLTVPGSERSYDLTGLKPGTEYTVSIYG
VVTALPSYYSSNPLSAIFTTGGHREIHHIIGGLPETGGH
SEQ ID no. 26 = ISOP130R5CP6_A06_Y25A
MLPAPKNLVVSRVTEDSARLSWKHARPGARFDSFLIQYQESEKVGEAIVLTVPGSERSYDLTGLKPGTEYTVSIYG
VVTALPSYYSSNPLSAIFTTGGHREIHHIIGGLPETGGH
SEQ ID no. 27 = ISOP130R5CP6_A06_R26A
MLPAPKNLVVSRVTEDSARLSWKHYAPGARFDSFLIQYQESEKVGEAIVLTVPGSERSYDLTGLKPGTEYTVSIYG
VVTALPSYYSSNPLSAIFTTGGHHHHHEGGLPETGGH
SEQ ID no. 28 = ISOP130R5CP6_A06_P27A
MLPAPKNLVVSRVTEDSARLSWKHYRAGARFDSFLIQYQESEKVGEAIVLTVPGSERSYDLTGLKPGTEYTVSIYG
VVTALPSYYSSNPLSAIFTTGGHHHHHEGGLPETGGH
SEQ ID no. 29 = ISOP130R5CP6_A06_G28A
MLPAPKNLVVSRVTEDSARLSWKHYRPAARFDSFLIQYQESEKVGEAIVLTVPGSERSYDLTGLKPGTEYTVSIYG
VVTALPSYYSSNPLSAIFTTGGHREIHHIIGGLPETGGH
SEQ ID no. 30 = ISOP130R5CP6_A06_R30A
MLPAPKNLVVSRVTEDSARLSWKHYRPGAAFDSFLIQYQESEKVGEAIVLTVPGSERSYDLTGLKPGTEYTVSIYG
VVTALPSYYSSNPLSAIFTTGGHHHHIREIGGLPETGGH
SEQ ID no. 31 = ISOP130R5CP6_A06_V78A
MLPAPKNLVVSRVTEDSARLSWKHYRPGARFDSFLIQYQESEKVGEAIVLTVPGSERSYDLTGLKPGTEYTVSIYG
VATALPSYYSSNPLSAIFTTGGHREIHREIGGLPETGGH
SEQ ID no. 32 = ISOP130R5CP6_A06_T79A
MLPAPKNLVVSRVTEDSARLSWKHYRPGARFDSFLIQYQESEKVGEAIVLTVPGSERSYDLTGLKPGTEYTVSIYG
VVAALPSYYSSNPLSAIFTTGGHREIHREIGGLPETGGH
SEQ ID no. 33 = ISOP130R5CP6_A06_L81A
MLPAPKNLVVSRVTEDSARLSWKHYRPGARFDSFLIQYQESEKVGEAIVLTVPGSERSYDLTGLKPGTEYTVSIYG
VVTAAPSYYSSNPLSAIFTTGGHREIHREIGGLPETGGH
SEQ ID no. 34 = ISOP130R5CP6_A06_P82A
MLPAPKNLVVSRVTEDSARLSWKHYRPGARFDSFLIQYQESEKVGEAIVLTVPGSERSYDLTGLKPGTEYTVSIYG
VVTALASYYSSNPLSAIFTTGGHREIHREIGGLPETGGH
SEQ ID no. 35 = ISOP130R5CP6_A06_S83A
MLPAPKNLVVSRVTEDSARLSWKHYRPGARFDSFLIQYQESEKVGEAIVLTVPGSERSYDLTGLKPGTEYTVSIYG
VVTALPAYYSSNPLSAIFTTGGHHEIHREIGGLPETGGH
SEQ ID no. 36 = ISOP130R5CP6_A06_Y84A
MLPAPKNLVVSRVTEDSARLSWKHYRPGARFDSFLIQYQESEKVGEAIVLTVPGSERSYDLTGLKPGTEYTVSIYG
VVTALPSAYSSNPLSAIFTTGGHREIHREIGGLPETGGH
SEQ ID no. 37 = ISOP130R5CP6_A06_Y85A
MLPAPKNLVVSRVTEDSARLSWKHYRPGARFDSFLIQYQESEKVGEAIVLTVPGSERSYDLTGLKPGTEYTVSIYG
VVTALPSYASSNPLSAIFTTGGHREIHREIGGLPETGGH
SEQ ID no. 38 = ISOP130R5CP6_A06_S86A
MLPAPKNLVVSRVTEDSARLSWKHYRPGARFDSFLIQYQESEKVGEAIVLTVPGSERSYDLTGLKPGTEYTVSIYG
VVTALPSYYASNPLSAIFTTGGHHHHHHGGLPETGGH

1. A composition comprising a fibronectin type III (FN3) domain-nanoparticle complex, wherein the composition comprises a FN3 domain conjugated to a surface of a nanoparticle.

2. (canceled)

3. The composition of claim 1, wherein the nanoparticle is a lipid nanoparticle, Poly Lactic-co-Glycolic Acid (PLGA) nanoparticle, or a cyclodextrin polymeric nanoparticle (CDP).

4-6. (canceled)

7. The composition of claim 1, wherein the nanoparticle comprises a polynucleotide.

8-11. (canceled)

12. The composition of claim 1, wherein nanoparticle comprises an additional active agent selected from the group consisting of proteins, peptides, small molecule compounds, and immunostimulatory agents.

13. The composition of claim 1, wherein the FN3 domain binds to PSMA, EGFR, EpCam, CD22, BCMA, CD33, CD71 and/or CD8.

14-16. (canceled)

17. The composition of claim 7, wherein the polynucleotide is an antisense oligonucleotide (ASO) and the FN3 is a FN3 domain that binds to PSMA, EGFR, EpCam, CD22, BCMA, CD33, CD71 and/or CD8.

18-26. (canceled)

27. A composition comprising a fibronectin type III (FN3) domain conjugated to a conjugate, wherein the FN3 domain comprises a sequence of SEQ ID NOS: 1-6, 8-11, 14-38 or 40-46.

28. The composition of claim 27, wherein the conjugate is a toxin.

29-30. (canceled)

31. The composition of claim 27, wherein the FN3 domain binds to EpCAM.

32-33. (canceled)

34. A peptide comprising a sequence having at least 90% homology to a peptide having the sequence of SEQ ID NOS: 1-6, 8-11, 14-38 and 40-46.

35. The peptide of claim 34, wherein the peptide comprises a sequence of SEQ ID NOS: 1-6, 8-11, 14-38 and 40-46.

36. The peptide of claim 34, wherein the peptide consists a sequence of SEQ ID NOS: 1-6, 8-11, 14-38 and 40-46.

37. A peptide comprising a sequence of SEQ ID NOS: 1-6, 8-11, 14-38 and 40-46, wherein at least one residue is substituted with a cysteine at a position corresponding to a residue at a position of 6, 8, 10, 11, 14, 15, 16, 20, 30, 34, 38, 40, 41, 45, 47, 48, 53, 54, 59, 60, 62, 64, 70, 88, 89, 90, 91, and 93.

38. (canceled)

39. A method of targeting a cell expressing EpCAM, the method comprising contacting a cell with a FN3 domain that binds to EpCAM.

40. The method of claim 39, wherein the FN3 domain that binds to EpCAM is conjugated to a conjugate.

41. The method of claim 39, wherein the FN3 domain comprises a sequence of SEQ ID NO.: 14-38, or variants thereof.

42. The method of claim 40, wherein the conjugate is a surface of the nanoparticle.

44-48. (canceled)

49. The method of claim 40, wherein the nanoparticle comprises a polynucleotide.

50-53. (canceled)

54. The method of claim 41, wherein nanoparticle comprises an additional active agent selected from the group consisting of proteins, peptides, small molecule compounds, and immunostimulatory agents.

55-59. (canceled)

60. A method of treating cancer or an auto-immune disease in a patient, the method comprising administering a composition of claim 1 to the patient to treat the cancer or the auto-immune disease.

61-62. (canceled)


SEQUENCE LISTING (63 sequences)

SEQ ID NO: 1 - protein, 90 aa, Artificial Sequence
  P155R8-G3
MLPAPKNLVVSRVTEDSARLSWTAPDAAFDSFQIYYSELLSYGEAIVLTVPGSERSYDLTGLKPGTEYTVSINGVKGGTRSWSLSAIFTT

SEQ ID NO: 2 - protein, 250 aa, Artificial Sequence
  EEB8
MLPAPKNLVVSRVTEDSARLSWTAPDAAFDSFQIYYSELLSYGEAIVLTVPGSERSYDLTGLCPGTEYTVSINGVKGGTRSWSLSAIFTTAPAPAPAPAPLPAPKNLVVSRVTEDSARLSWTAPDAAFDSFQIYYSELLSYGEAIVLTVPGSERSYDLTGLKPGTEYTVSINGVKGGTRSWSLSAIFTTAPAPAPAPAPTIDEWLLKEAKEKAIEELKKAGITSDYYFDLINKAKTVEGVNALKDEILKA

SEQ ID NO: 3 - protein, 250 aa, Artificial Sequence
  EEB9
MLPAPKNLVVSRVTEDSARLSWTAPDAAFDSFQIYYSELLSYGEAIVLTVPGSERSYDLTGLCPGTEYTVSINGVKGGTRSWSLSAIFTTAPAPAPAPAPLPAPKNLVVSRVTEDSARLSWTAPDAAFDSFQIYYSELLSYGEAIVLTVPGSERSYDLTGLCPGTEYTVSINGVKGGTRSWSLSAIFTTAPAPAPAPAPTIDEWLLKEAKEKAIEELKKAGITSDYYFDLINKAKTVEGVNALKDEILKA

SEQ ID NO: 4 - protein, 250 aa, Artificial Sequence
  EEB11
MLPAPKNLVVSRVTEDSARLSWTAPDAAFDSFQIYYSELLSYGEAIVLTVPGSERSYDLTGLCPGTEYTVSINGVKGGTRSWSLSAIFTTAPCPAPAPAPLPAPKNLVVSRVTEDSARLSWTAPDAAFDSFQIYYSELLSYGEAIVLTVPGSERSYDLTGLCPGTEYTVSINGVKGGTRSWSLSAIFTTAPAPAPAPAPTIDEWLLKEAKEKAIEELKKAGITSDYYFDLINKAKTVEGVNALKDEILKA

SEQ ID NO: 5 - protein, 250 aa, Artificial Sequence
  EEB10
MLPAPKNLVVSRVTEDSARLSWTAPDAAFDSFQIYYSELLSYGEAIVLTVPGSERSYDLTGLCPGTEYTVSINGVKGGTRSWSLSAIFTTAPCPAPAPAPLPAPKNLVVSRVTEDSARLSWTAPDAAFDSFQIYYSELLSYGEAIVLTVPGSERSYDLTGLCPGTEYTVSINGVKGGTRSWSLSAIFTTAPCPAPAPAPTIDEWLLKEAKEKAIEELKKAGITSDYYFDLINKAKTVEGVNALKDEILKA

SEQ ID NO: 6 - protein, 250 aa, Artificial Sequence
  ECB166
MLPAPKNLVVSEVTEDSARLSWTAPDAAFDSFLIQYQESEKVGEAIVLTVPGSERSYDLTGLCPGTEYTVSIYGVKGGHRSNPLSAIFTTAPCPAPAPAPLPAPKNLVVSEVTEDSARLSWTAPDAAFDSFLIQYQESEKVGEAIVLTVPGSERSYDLTGLCPGTEYTVSIYGVKGGHRSNPLSAIFTTAPAPAPAPAPTIDEWLLKEAKEKAIEELKKAGITSDYYFDLINKAKTVEGVNALKDEILKA

SEQ ID NO: 7 - protein, 51 aa, Artificial Sequence
  ABDcon12
TIDEWLLKEAKEKAIEELKKAGITSDYYFDLINKAKTVEGVNALKDEILKA

SEQ ID NO: 8 - protein, 89 aa, Artificial Sequence
  P233FR9P1001_H3-1
LPAPKNLVVSRVTEDSARLSWTAPDAAFDSFPIGYWEWDDDGEAIVLTVPGSERSYDLTGLKPGTEYHVYIAGVKGGQWSFPLSAIFTT

SEQ ID NO: 9 - protein, 95 aa, Artificial Sequence
  P234CR9_H01
LPAPKNLVVSRVTEDSARLSWEWWVIPGDFDSFLIQYQESEKVGEAIVLTVPGSERSYDLTGLKPGTEYTVSIYGVVNSGQWNDTSNPLSAIFTT

SEQ ID NO: 10 - protein, 89 aa, Artificial Sequence
  P233FR9_H10
LPAPKNLVVSRVTEDSARLSWTAPDAAFDSFAIGYWEWDDDGEAIVLTVPGSERSYDLTGLKPGTEYPVYIAGVKGGQWSFPLSAIFTT

SEQ ID NO: 11 - protein, 92 aa, Artificial Sequence
  P229CR5P819_H11
LPAPKNLVVSRVTEDSARLSWDIDEQRDWFDSFLIQYQESEKVGEAIVLTVPGSERSYDLTGLKPGTEYTVSIYGVYHVYRSSNPLSAIFTT

SEQ ID NO: 12 - protein, 199 aa, Artificial Sequence
  Sortase A
MSHHHHHHSSGENLYFQSKPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVK

SEQ ID NO: 13 - protein, 182 aa, Artificial Sequence
  Tagless Sortase A
SKPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVK

SEQ ID NO: 14 - protein, 107 aa, Artificial Sequence
  ISOP130R5CP6_A06_cys
MLPAPKNLVVSRVTEDSARLSWKHYRPGARFDSFLIQYQESEKVGEAIVLTVPGSERSYDLTGLKPGTEYTVSIYGVVTALPSYYSSNPLSAIFTTGGHHHHHHGGC

SEQ ID NO: 15 - protein, 107 aa, Artificial Sequence
  ISOP130R5CP7_G04_cys
MLPAPKNLVVSRVTEDSARLSWKHYRPGARFDSFLIQYQESEKVGEAIVLTVPGSERSYDLTGLKPGTEYTVSIYGVAAHAIPRYASNPLSAIFTTGGHHHHHHGGC

SEQ ID NO: 16 - protein, 105 aa, Artificial Sequence
  ISO124R5AB_D6_cys
MLPAPKNLVVSRVTEDSARLSWHNHRPQFDSFLIQYQESEKVGEAIVLTVPGSERSYDLTGLKPGTEYTVSIYGVAIAVPWNYQSNPLSAIFTTGGHHHHHHGGC

SEQ ID NO: 17 - protein, 114 aa, Artificial Sequence
  ISOP130R5CP7_G02srt
MLPAPKNLVVSRVTEDSARLSWKHYRPGARFDSFLIQYQESEKVGEAIILTVPGSERSYDLTGLKPGTEYTVSIYGVVTHALPTAYTSNPLSAIFTTGGHHHHHHGGLPETGGH

SEQ ID NO: 18 - protein, 113 aa, Artificial Sequence
  ISOP130R5CP6_E08srt
MLPAPKNLVVSRVTEDSARLSWKHYRPGARFDSFLIQYQESEKVGEAIVLTVPGSERSYDLTGLKPGTEYTVSIYGVVAALPNNYASNPLSAIFTTGGHHHHHHGGLPETGGH

SEQ ID NO: 19 - protein, 113 aa, Artificial Sequence
  ISOP130R5CP6_A06srt
MLPAPKNLVVSRVTEDSARLSWKHYRPGARFDSFLIQYQESEKVGEAIVLTVPGSERSYDLTGLKPGTEYTVSIYGVVTALPSYYSSNPLSAIFTTGGHHHHHHGGLPETGGH

SEQ ID NO: 20 - protein, 113 aa, Artificial Sequence
  ISOP130R5CP6_F01srt
MLPAPKNLVVSRVTEDSARLSWKHYRPGARFDSFLIQYQESEKVGEAIVLTVPGSERSYDLTGLKPGTEYTVSIYGVVTALPSNYISNPLSAIFTTGGHHHHHHGGLPETGGH

SEQ ID NO: 21 - protein, 114 aa, Artificial Sequence
  ISOP130R5CP6_F02srt
MLPAPKNLVVSRVTEDSARLSWDQYRKYAGFDSFLIQYQESEKVGEAIVLTVPGSERSYDLTGLKPGTEYTVSIYGVVTHALPQTYQSNPLSAIFTTGGHHHHHHGGLPETGGH

SEQ ID NO: 22 - protein, 114 aa, Artificial Sequence
  ISOP130R5CP7_F11srt
MLPAPKNLVVSRVTEDSARLSWKHYRPGARFDSFLIQYQESEKVGEAIVLTVPGSERSYDLTGLKPGTEYTVSIYGVIWGALPNSYSSNPLSAIFTTGGHHHHHHGGLPETGGH

SEQ ID NO: 23 - protein, 114 aa, Artificial Sequence
  ISOP130R5CP6_A02srt
MLPAPKNLVVSRVTEDSARLSWKHYRPGARFDSFLIQYQESEKVGEAIVLTVPGSERSYDLTGLKPGTEYTVSIYGVVNNALPRWYISNPLSAIFTTGGHHHHHHGGLPETGGH

SEQ ID NO: 24 - protein, 113 aa, Artificial Sequence
  ISOP130R5CP6_A06_K23A
MLPAPKNLVVSRVTEDSARLSWAHYRPGARFDSFLIQYQESEKVGEAIVLTVPGSERSYDLTGLKPGTEYTVSIYGVVTALPSYYSSNPLSAIFTTGGHHHHHHGGLPETGGH

SEQ ID NO: 25 - protein, 113 aa, Artificial Sequence
  ISOP130R5CP6_A06_H24A
MLPAPKNLVVSRVTEDSARLSWKAYRPGARFDSFLIQYQESEKVGEAIVLTVPGSERSYDLTGLKPGTEYTVSIYGVVTALPSYYSSNPLSAIFTTGGHHHHHHGGLPETGGH

SEQ ID NO: 26 - protein, 113 aa, Artificial Sequence
  ISOP130R5CP6_A06_Y25A
MLPAPKNLVVSRVTEDSARLSWKHARPGARFDSFLIQYQESEKVGEAIVLTVPGSERSYDLTGLKPGTEYTVSIYGVVTALPSYYSSNPLSAIFTTGGHHHHHHGGLPETGGH

SEQ ID NO: 27 - protein, 113 aa, Artificial Sequence
  ISOP130R5CP6_A06_R26A
MLPAPKNLVVSRVTEDSARLSWKHYAPGARFDSFLIQYQESEKVGEAIVLTVPGSERSYDLTGLKPGTEYTVSIYGVVTALPSYYSSNPLSAIFTTGGHHHHHHGGLPETGGH

SEQ ID NO: 28 - protein, 113 aa, Artificial Sequence
  ISOP130R5CP6_A06_P27A
MLPAPKNLVVSRVTEDSARLSWKHYRAGARFDSFLIQYQESEKVGEAIVLTVPGSERSYDLTGLKPGTEYTVSIYGVVTALPSYYSSNPLSAIFTTGGHHHHHHGGLPETGGH

SEQ ID NO: 29 - protein, 113 aa, Artificial Sequence
  ISOP130R5CP6_A06_G28A
MLPAPKNLVVSRVTEDSARLSWKHYRPAARFDSFLIQYQESEKVGEAIVLTVPGSERSYDLTGLKPGTEYTVSIYGVVTALPSYYSSNPLSAIFTTGGHHHHHHGGLPETGGH

SEQ ID NO: 30 - protein, 113 aa, Artificial Sequence
  ISOP130R5CP6_A06_R30A
MLPAPKNLVVSRVTEDSARLSWKHYRPGAAFDSFLIQYQESEKVGEAIVLTVPGSERSYDLTGLKPGTEYTVSIYGVVTALPSYYSSNPLSAIFTTGGHHHHHHGGLPETGGH

SEQ ID NO: 31 - protein, 113 aa, Artificial Sequence
  ISOP130R5CP6_A06_V78A
MLPAPKNLVVSRVTEDSARLSWKHYRPGARFDSFLIQYQESEKVGEAIVLTVPGSERSYDLTGLKPGTEYTVSIYGVATALPSYYSSNPLSAIFTTGGHHHHHHGGLPETGGH

SEQ ID NO: 32 - protein, 113 aa, Artificial Sequence
  ISOP130R5CP6_A06_T79A
MLPAPKNLVVSRVTEDSARLSWKHYRPGARFDSFLIQYQESEKVGEAIVLTVPGSERSYDLTGLKPGTEYTVSIYGVVAALPSYYSSNPLSAIFTTGGHHHHHHGGLPETGGH

SEQ ID NO: 33 - protein, 113 aa, Artificial Sequence
  ISOP130R5CP6_A06_L81A
MLPAPKNLVVSRVTEDSARLSWKHYRPGARFDSFLIQYQESEKVGEAIVLTVPGSERSYDLTGLKPGTEYTVSIYGVVTAAPSYYSSNPLSAIFTTGGHHHHHHGGLPETGGH

SEQ ID NO: 34 - protein, 113 aa, Artificial Sequence
  ISOP130R5CP6_A06_P82A
MLPAPKNLVVSRVTEDSARLSWKHYRPGARFDSFLIQYQESEKVGEAIVLTVPGSERSYDLTGLKPGTEYTVSIYGVVTALASYYSSNPLSAIFTTGGHHHHHHGGLPETGGH

SEQ ID NO: 35 - protein, 113 aa, Artificial Sequence
  ISOP130R5CP6_A06_S83A
MLPAPKNLVVSRVTEDSARLSWKHYRPGARFDSFLIQYQESEKVGEAIVLTVPGSERSYDLTGLKPGTEYTVSIYGVVTALPAYYSSNPLSAIFTTGGHHHHHHGGLPETGGH

SEQ ID NO: 36 - protein, 113 aa, Artificial Sequence
  ISOP130R5CP6_A06_Y84A
MLPAPKNLVVSRVTEDSARLSWKHYRPGARFDSFLIQYQESEKVGEAIVLTVPGSERSYDLTGLKPGTEYTVSIYGVVTALPSAYSSNPLSAIFTTGGHHHHHHGGLPETGGH

SEQ ID NO: 37 - protein, 113 aa, Artificial Sequence
  ISOP130R5CP6_A06_Y85A
MLPAPKNLVVSRVTEDSARLSWKHYRPGARFDSFLIQYQESEKVGEAIVLTVPGSERSYDLTGLKPGTEYTVSIYGVVTALPSYASSNPLSAIFTTGGHHHHHHGGLPETGGH

SEQ ID NO: 38 - protein, 113 aa, Artificial Sequence
  ISOP130R5CP6_A06_S86A
MLPAPKNLVVSRVTEDSARLSWKHYRPGARFDSFLIQYQESEKVGEAIVLTVPGSERSYDLTGLKPGTEYTVSIYGVVTALPSYYASNPLSAIFTTGGHHHHHHGGLPETGGH

SEQ ID NO: 39 - protein, 89 aa, Artificial Sequence
  Tencon
LPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 40 - protein, 103 aa, Artificial Sequence
  83v10
MLPAPKNLVVSEVTCDSARLSWDDPWAFYESFLIQYQESEKVGEAIVLTVPGSERSYDLTGLKPGTEYTVSIYGVHNVYKDTNMRGLPLSAIFTTGGHHHHHH

SEQ ID NO: 41 - protein, 103 aa, Artificial Sequence
  83v11
MLPAPKNLVVSCVTEDSARLSWDDPWAFYESFLIQYQESEKVGEAIVLTVPGSERSYDLTGLKPGTEYTVSIYGVHNVYKDTNMRGLPLSAIFTTGGHHHHHH

SEQ ID NO: 42 - protein, 103 aa, Artificial Sequence
  83v12
MLPAPKNLVVSEVTEDSACLSWDDPWAFYESFLIQYQESEKVGEAIVLTVPGSERSYDLTGLKPGTEYTVSIYGVHNVYKDTNMRGLPLSAIFTTGGHHHHHH

SEQ ID NO: 43 - protein, 103 aa, Artificial Sequence
  83v13
MLPAPKNLVVSEVTEDSARLSWDDPWAFYESFLIQYQESEKVGEAIVLTVPGSCRSYDLTGLKPGTEYTVSIYGVHNVYKDTNMRGLPLSAIFTTGGHHHHHH

SEQ ID NO: 44 - protein, 103 aa, Artificial Sequence
  83v14
MLPAPKNLVVSEVTEDSARLSWDDPWAFYESFLIQYQESEKVGEAIVLTVPGSERSYDLTGLCPGTEYTVSIYGVHNVYKDTNMRGLPLSAIFTTGGHHHHHH

SEQ ID NO: 45 - protein, 104 aa, Artificial Sequence
  83v15
MLPAPKNLVVSEVTEDSARLSWDDPWAFYESFLIQYQESEKVGEAIVLTVPGSERSYDLTGLKPGTEYTVSIYGVHNVYKDTNMRGLPLSAIFTTGGHHHHHHC

SEQ ID NO: 46 - protein, 99 aa, Artificial Sequence
  G3
MLPAPKNLVVSRVTEDSARLSWTAPDAAFDSFQIYYSELLSYGEAIVLTVPGSERSYDLTGLKPGTEYTVSINGVKGGTRSWSLSAIFTTGGHHHHHHC

SEQ ID NO: 47 - DNA, 20 bp, Artificial Sequence
  MALATI-Seq 1
  modified_base (1)..(5): 2′ O methyl
  modified_base (1)..(20): phosphorothioate linkage
  modified_base (16)..(20): 2′ O methyl
ugccuttaggattctagaca

SEQ ID NO: 48 - DNA, 20 bp, Artificial Sequence
  MALATI-Seq 2
  modified_base (1)..(5): 2′ O methyl
  modified_base (1)..(20): phosphorothioate linkage
  modified_base (16)..(20): 2′ O methyl
ccaggctggttatgacucag

SEQ ID NO: 49 - protein, 97 aa, Artificial Sequence
  Tagless 83v10
MLPAPKNLVVSEVTCDSARLSWDDPWAFYESFLIQYQESEKVGEAIVLTVPGSERSYDLTGLKPGTEYTVSIYGVHNVYKDTNMRGLPLSAIFTTGG

SEQ ID NO: 50 - protein, 7 aa, Artificial Sequence
  FG Loop
KGGHRSN

SEQ ID NO: 51 - protein, 8 aa, Artificial Sequence
  BC Loop
TAPDAAFD

SEQ ID NO: 52 - protein, 8 aa, Artificial Sequence
  BC Loop
KHYRPGAR

SEQ ID NO: 53 - protein, 6 aa, Artificial Sequence
  BC Loop
HNHRPQ

SEQ ID NO: 54 - protein, 11 aa, Artificial Sequence
  FG Loop
VTALPSYYSSN

SEQ ID NO: 55 - protein, 11 aa, Artificial Sequence
  FG Loop
AAHAIPRYASN

SEQ ID NO: 56 - protein, 11 aa, Artificial Sequence
  FG Loop
AIAVPWNYQSN

SEQ ID NO: 57 - protein, 8 aa, Artificial Sequence
  BC Loop
DQYRKYAG

SEQ ID NO: 58 - protein, 12 aa, Artificial Sequence
  FG Loop
VTHALPTAYTSN

SEQ ID NO: 59 - protein, 11 aa, Artificial Sequence
  FG Loop
VAALPNNYASN

SEQ ID NO: 60 - protein, 12 aa, Artificial Sequence
  FG Loop
VTHALPQTYQSN

SEQ ID NO: 61 - protein, 12 aa, Artificial Sequence
  FG Loop
IWGALPNSYSSN

SEQ ID NO: 62 - protein, 11 aa, Artificial Sequence
  FG Loop
VTALPSNYISN

SEQ ID NO: 63 - protein, 12 aa, Artificial Sequence
  FG Loop
VNNALPRWYISN